(19) United States
(12) Patent Application Publication
(10) Pub. No.: US 2026/0053367 A1
(43) Pub. Date: Feb. 26, 2026

(54) SYSTEMS AND METHODS FOR FLUORESCENCE IMAGING IN THE VISIBLE BAND

(71) Applicant: Stryker Corporation, Portage, MI (US)

(72) Inventors: Benjamin Hyman FEINGOLD, San Francisco, CA (US); Marc ANDRÉ, Spiegel b. Bern (CH); Paul Roald WESTWICK, Vancouver (CA); Robert Anthony STEAD, North Vancouver (CA); Eric Charles HUYNH, San Ramon, CA (US); Donna HOHERTZ, Vancouver (CA)

(73) Assignee: Stryker Corporation, Portage, MI (US)

(21) Appl. No.: 19/310,520

(22) Filed: Aug. 26, 2025

Related U.S. Application Data
(60) Provisional application No. 63/687,209, filed on Aug. 26, 2024.

(51) Int. Cl. A61B 5/00; G06T 11/00

(52) U.S. Cl. CPC A61B 5/0071; G06T 11/001; A61B 2560/0223

(57) ABSTRACT

Techniques for fluorescence imaging are provided. A tissue region comprising a target fluorophore is illuminated with an excitation light in a wavelength range of 380-490 nm. First image data of the tissue is captured at an image sensor, wherein the first image comprises an autofluorescence contribution from the tissue and a fluorescence emission contribution from the target fluorophore, wherein the first image data comprises red channel data and green channel data. A corrected fluorescence image is generated, based on the first image data, wherein generating the fluorescence image comprises subtracting a second component based on one of the green channel data or the red channel data from a first component based on the other of the green channel data or the red channel data.

### CROSS-REFERENCE TO RELATED APPLICATIONS

This application claims the benefit of U.S. Provisional Application No. 63/687,209, filed Aug. 26, 2024, the entire contents of which is incorporated herein by reference.

### FIELD

This relates generally to imaging techniques for fluorescence imaging in the visible band, and more specifically to systems and methods for imaging using fluorophores that excite in the UV, violet, and/or blue range and that emit in the visible range.

### BACKGROUND

Fluorescence imaging is a common technique in modern medical imaging. Different fluorophores used in medical imaging excite and emit in different characteristic wavelength ranges. Certain fluorophores used in medical imaging excite in the UV, violet, and/or blue range of about 380-490 nm and emit in the visible light range. For example, the 5-ala fluorophore (Pp-IX) has an absorption peak of about 410 nm (and absorbs at over 90% between 400 and 420 nm) and emits at an emission range of about 600-720 nm, with an emission peak at about 635 nm.

### SUMMARY

For fluorescence medical imaging using fluorophores that excite in the UV, violet, and/or blue range and emit at a visible-light emission rage, background autofluorescence may be present, because many tissues and proteins show autofluorescence when illuminated with light at wavelengths of less than about 550 nm. This autofluorescence occurs primarily in the blue-green range of about 425-575 nm. Background autofluorescence makes it difficult to effectively resolve regions of the tissue where the exogenous fluorophore to be imaged (e.g., the imaging agent) is actually present.

Known solutions to address background autofluorescence when using these fluorophores include (a) using one or more filters to image light only at a narrow band around an emission peak of the fluorophore and/or (b) blocking the autofluorescence in the blue spectrum by blocking that part of the visible spectrum. However, these techniques have several drawbacks. For example, use of a filter limits an imaging system to a specific fluorescence agent having the emission peak or spectrum for which the filter is selected, making such a system unsuitable for cross-specialty visualization. Specifically, there are issues with autofluorescence as well as with simultaneous imaging of white light and those visible fluorophores. Solutions for manually or robotically inserting and removing different filters are cumbersome, time-consuming, expensive, and failure-prone. Furthermore, blocking of autofluorescence requires blocking of visible light wavelengths, therefore precluding the system from providing white light imaging capabilities.

Accordingly, there is a need for improved systems and methods for fluorescence imaging in the visible band, including techniques that allow for excitation in the UV, violet, and/or blue range and that effectively distinguish fluorescence emission of fluorescence agents in the visible light range from autofluorescence, without reliance on filtering principles that preclude white light imaging. Particularly, there is a need for improved systems and methods for fluorescence imaging in the visible band that allow for the use of the same or similar imaging modalities as those afforded by the use of indocyanine green (ICG). Disclosed herein are systems and methods that may address one or more of the above-identified needs.

In some aspects, a system for fluorescence imaging in the visible band as disclosed herein reduces the effects of background autofluorescence by subtracting a scaled amount of the green image signal (and, optionally, a scaled amount of the blue image signal) from the red (visible fluorophore emission) image signal.

As described herein, separation of signal due to fluorescence of an exogenous fluorophore (e.g., imaging agent) and signal due to autofluorescence is achieved by leveraging the spectral separation between the primary emission band of the fluorescence signal and the primary emission band of the autofluorescence. By capturing these two bands with different color channels, and then applying appropriate image processing, the signals from the two different bands can be effectively resolved from one another, allowing an image clearly showing the fluorescence agent in the tissue to be generated.

Furthermore, when performing fluorescence imaging using fluorescence imaging agents that excite in a UV, violet, and/or blue range and emit in a visible light range, it may be desirable in some aspects to adjust a visible light exposure between capturing different images, for example, between capturing different images in a time series. However, adjusting a pulse width of a visible light illumination light between different images, in order to achieve the desired white light exposure adjustment, may also cause undesirable changes in white balance between the two images. This is because some reflected illumination light may be captured in a blue channel of the captured visible light image, so the later-captured image(s) may be shifted towards the blue as compared to the earlier-captured image(s). Thus, there is a need for imaging techniques for adjusting white light exposure of different images while illuminating a fluorescence agent in the UV, violet, and/or blue range, while maintaining white balance of the captured images.

Disclosed herein are one or more techniques that may address the above-identified need. In some aspects, one or more adjustments to imaging settings—including illumination settings, capture settings, image generation settings, and/or image post-processing settings—may be applied such that white light exposure of different images can vary with respect to one another while white balance of the different images is held constant (or nearly constant).

Furthermore, aspects in which no optical filter is used for collection may be advantageous. For example, imaging techniques that do not require use of, and possible manual insertion of, an optical filter may simplify operation and manufacture and may reduce opportunities for user error during imaging. However, when no optical filter is used for collection, UV, violet, and/or blue illumination light that is used to excite fluorophores may “leak” into the red pixels on a Bayer sensor, or into the red channel of an RGB prism camera, because these red pixels and/or red channel of an RGB prism may have a non-zero response to short wavelength excitation light (e.g., in the 390-425 nm range), and this effect may become more critical when the excitation light is bright and not blocked from the image sensor. Generally speaking, because of the imperfect nature of image sensors and camera design, UV, violet, and/or blue light may cross-contaminate (leak onto) pixels/sensors/channels designed to detect other parts of the optical spectrum (for example green and red). Thus, an optical channel configured to image in the visible light range may detect UV light and may, by design, detect violet and/or blue light via its blue light pixels/channels, particularly in the wavelength range of about 400-450 nm; the channel may also detect UV, violet, and/or blue light via its red and green light pixels/channels particularly in the wavelength range of about 400-425 nm.

Accordingly, there is a need for improved systems and methods for fluorescence imaging in the visible band, including techniques that allow for excitation in the UV, violet, and/or blue range and that effectively account for leakage of UV, violet, and/or blue excitation light into the visible light range, without reliance on use of optical filters for collection. Disclosed herein are systems and methods that may address one or more of the above-identified needs.

In some aspects, filterless fluorescence imaging with illumination in the UV, violet, and/or blue range and imaging in the white light range is enabled by illuminating tissue with UV, violet, and/or blue illumination, collecting fluorescence imaging data using a red channel (e.g., a red sensor of an RGB prism arrangement, or a red pixel of a Bayer array sensor), and collecting background imaging data using one or both of a blue channel and a green channel. In some aspects, background imaging data may be collected using a blue channel only (and, e.g., the green channel may be ignored); in some aspects, background imaging data may be collected using both the blue and green channel.

In some aspects, a Blue-Green offset (BG offset) may be applied. In some aspects, more BG offset may be applied to allow for large signal difference between blue and red. The offset may be chosen such that the blue and/or green channel have a balanced exposure (highlights are not blown out and detail is retained in the shadows) and the red (fluorescence) channel is at the user desired intensity to see a feature of interest.

In some aspects using an RGB prism implementation, the three sensors may be running at different gains and/or different exposure periods. An effective difference in gain (combined gain and exposure) may determine the fluorescence sensitivity. In cases in which no excitation-blocking filter is used, it may be desirable for that difference to be greater by about 30 dB or 32 times. For example, in implementations that use a filter, a user may be able to adjust offset from +0 dB to +27 dB; however, in implementations that do not use a filter, an adjustable range for offset may be restricted to about +30 dB to about +57 dB, combined gain and exposure length difference. While the description herein may refer to exposure lengths for different sensors relative to one another, and may refer to exposure length ratios for different sensors, it should be understood that these examples of exposure lengths may or may not be implemented with different gains for the different sensors. In some aspects, the exemplary relative comparisons and ratios given for different exposure lengths for different sensors may also apply equally to relative comparisons and ratios for effective gain, which may be a combination (e.g., a product) of exposure length and gain, for different sensors. For example, while the specification may refer to an example in which a red-channel exposure length is 10× longer than a blue-channel exposure length, it should be understood that in some aspects the system may implement the same 10× ratio for the effective gain of the red channel to the blue channel using different exposure length ratios (for example using a 5×-longer red-channel exposure length with a 2× greater gain applied to the red channel, or alternatively using the same exposure length for the red channel a blue channel but applying a 10× gain to the red channel).

In some aspects, different exposure lengths per frame may be used for the red channel, green channel, and/or blue channel. In some aspects, the exposure of the blue and/or green channels may be about 1/10 or less per frame than the exposure of the red channel. In some aspects, the exposure of the blue and/or green channels may be about 1/25 or less per frame than the exposure of the red channel. In some aspects, the exposure of the blue channel may be about 1/25 or less per frame than the exposure of the red channel, and the exposure of the green channel may be about 1/10 or less per frame than the exposure of the red channel.

A difference may then be calculated based on at least the red channel data and the blue channel data (and optionally the green channel data) to generate a corrected fluorescence image based on the calculated difference. The corrected fluorescence image may thus provide a corrected image, based on the background imaging data, that corrects for leakage of the UV, violet, and/or blue illumination light into the red channel of the visible light sensor.

In some aspects, the above-described technique, of collecting fluorescence imaging data using a red channel and background imaging data using a blue channel and optionally a green channel, may be applied in a time-splicing schema. In these aspects, UV, violet, and/or blue illumination may be pulsed alternately with pulsed white-light illumination. The collection using the red channel for fluorescence imaging data and the blue and/or green channels for back ground imaging data (optionally, with different exposures for the different sensors) may be performed during UV, violet, and/or blue illumination pulses, while collection of white-light color-image data may be performed using all three channels of the white-light sensor (optionally, with equal exposures to one another) during pulses of the white-light illumination. This may allow for both a corrected fluorescence image and a color image to be generated, optionally for overlaid display with one another.

In some aspects, a Bayer array sensor may be used. This may avoid the need for use of a prism, which may allow for more compact devices.

In some aspects using a Bayer array sensor, continuous UV, violet, and/or blue illumination may be applied, fluorescence imaging data may be collected in fluorescence frames using only red pixels of the Bayer array sensor and using a first exposure for the red pixels, and background frames may be collected using only the blue (and optionally the green) pixels, and using exposure(s) that is/are less than the first exposure. Corrected background frames may be generated by adjusting color balance. Corrected fluorescence images may then be generated, which correct for UV, violet, and/or blue illumination light leakage into the red channel of the visible light sensor range, based on the fluorescence frames and on the corrected background frames.

In some aspects using a Bayer array sensor, UV, violet, and/or blue illumination may be pulsed with white-light illumination. During periods of UV, violet, and/or blue illumination, fluorescence frames may be collected using red pixels of the Bayer array sensor at a first exposure. During periods of UV, violet, and/or blue illumination, optionally, correction frames may be collected using blue pixels of the Bayer away sensor and using a second exposure shorter than the first exposure. During periods of white light illumination, optionally, white-light frames may be collected using red, green, and blue pixels of the Bayer array sensor and using a third exposure (which may optionally be shorter than the first exposure). Optionally, the white light frames may be used for automatic gain control. Optionally, the white light frames may be used to spoof G/B background. Optionally, corrected fluorescence frames may be generated by performing background subtraction based on the fluorescence frames and the background frames and/or based on spoofed G/B background from the white-light frames. Optionally, color overlay image data may be generated based on the corrected fluorescence frames and the white-light frames.

In some aspects, a first method for fluorescence imaging is provided, the first method comprising illuminating a tissue region with an excitation light, wherein the tissue region comprises tissue and a target fluorophore and wherein the excitation light comprises light within a wavelength range of 380-490 nm, capturing first image data of the tissue at an image sensor (or otherwise receiving the first image data), wherein the first image comprises an autofluorescence contribution from the tissue and a fluorescence emission contribution from the target fluorophore, wherein the first image data comprises red channel data and green channel data; and generating a corrected fluorescence image, based on the first image data, wherein generating the fluorescence image comprises subtracting a second component based on one of the green channel data or the red channel data from a first component based on the other of the green channel data or the red channel data.

In some aspects of the first method, the second component comprises a first weight.

In some aspects, the first method comprises selecting the first weight to minimize intensity of the fluorescence image in one or more regions of the fluorescence image in which the autofluorescence contribution is present but the fluorescence emission contribution is not present.

In some aspects, the first method comprises, before subtracting the second component from the first component, applying one or more preprocessing operations to one or both of the red channel data and the green channel data.

In some aspects of the first method, the second component is based on the red channel data and the first component is based on the green channel data.

In some aspects of the first method, the second component is based on the green channel data and the first component is based on the red channel data.

In some aspects of the first method: the first image data comprises blue channel data; and the second component is computed by subtracting a second sub-component based on the blue channel data from a first sub-component based on one of the green channel data or the red channel data.

In some aspects of the first method, the second sub-component comprises a second weight.

In some aspects, the first method comprises selecting the second weight to minimize an effect on the fluorescence image of increasing or decreasing an amount of blue light.

In some aspects, the first method comprises, before subtracting the second sub-component from the first sub-component, applying one or more preprocessing operations to the blue channel data.

In some aspects of the first method, capturing the first image data comprises exposing a red color channel, a green color channel, and a blue color channel simultaneously.

In some aspects, the first method comprises displaying an output image, wherein displaying the output is based at least in part on the generated corrected fluorescence image.

In some aspects, the first method comprises generating the output image, wherein generating the output image comprises: generating an uncorrected fluorescence image based on the first image data; and colorizing the corrected fluorescence image based on the uncorrected fluorescence image, wherein the colorization distinguishes the autofluorescence contribution from the fluorescence emission contribution.

In some aspects of the first method, generating the uncorrected fluorescence image comprises summing a fourth component based on one of the green channel data or the red channel data with a third component based on the other of the green channel data or the red channel data.

In some aspects, a first system for fluorescence imaging is provided, the first system comprising: an excitation light that illuminates a tissue region with an excitation light, wherein the tissue region comprises tissue and a target fluorophore and wherein the excitation light comprises light within a wavelength range of 380-490 nm; an image sensor that captures first image data of the tissue, wherein the first image comprises an autofluorescence contribution from the tissue and a fluorescence emission contribution from the target fluorophore, wherein the first image data comprises red channel data and green channel data; and one or more processors, coupled to the image sensor, that execute instructions stored in memory to generate a corrected fluorescence image, based on the first image data, wherein generating the fluorescence image comprises subtracting a second component based on one of the green channel data or the red channel data from a first component based on the other of the green channel data or the red channel data.

In some aspects, a first non-transitory computer-readable medium storing instructions for fluorescence imaging is provided, wherein the instructions, when executed by one or more processors of a system comprising an excitation light and an image sensor, cause the system to: illuminate, by the excitation light, a tissue region with an excitation light, wherein the tissue region comprises tissue and a target fluorophore and wherein the excitation light comprises light within a wavelength range of 380-490 nm; capture, by the image sensor, first image data of the tissue, wherein the first image comprises an autofluorescence contribution from the tissue and a fluorescence emission contribution from the target fluorophore, wherein the first image data comprises red channel data and green channel data; and generate, by the one or more processors, a corrected fluorescence image, based on the first image data, wherein generating the fluorescence image comprises subtracting a second component based on one of the green channel data or the red channel data from a first component based on the other of the green channel data or the red channel data.

In some aspects, a second method for white light and fluorescence imaging using visible light excitation is provided, the second method comprising: illuminating a tissue with a white light illumination light and with an excitation light, wherein the excitation light comprises light within a wavelength range of 380-490 nm; generating a first visible light image of the tissue based on first image data captured at an image sensor, wherein: a blue channel of the first visible light image includes a first white light contribution from reflected white light illumination light and a first excitation contribution from reflected excitation light, and the first white light contribution and the first excitation contribution define a first ratio; and after generating the first visible light image, generating a second visible light image of the tissue based on second image data captured at the image sensor, wherein: a white light exposure of the second visible light image is less than or equal to 90% of a white light exposure of the first image or is greater than or equal to 110% of the white light exposure of the first image, a blue channel of the second visible light image includes a second white light contribution from the reflected white light illumination light and a second excitation contribution from reflected excitation light, and the second white light contribution and the second excitation contribution define a second ratio that is greater than or equal to 95% of the first ratio and less than or equal to 105% of the first ratio.

In some aspects, the second method comprises, before generating the second visible light image: adjusting a gain of the image sensor to achieve the adjusted white light exposure of the second visible light image.

In some aspects, the second method comprises, before generating the second visible light image: adjusting an intensity of the white light illumination to achieve the adjusted white light exposure of the second visible light image.

In some aspects, the second method comprises, before generating the second visible light image: adjusting a white light pulse width of the white light illumination light to achieve the adjusted white light exposure of the second visible light image; and adjusting an intensity of the excitation light.

In some aspects, the second method comprises, before adjusting the intensity of the excitation light: calculating the first ratio for the first visible light image; and determining an amount by which to adjust the intensity of the excitation light based at least in part on the calculated ratio and on an amount by which the white light pulse width is adjusted.

In some aspects, the second method comprises, before adjusting the white light pulse width of the white light illumination, determining an amount by which to adjust the white light pulse width based at least in part on a distance between the image sensor and the tissue.

In some aspects, the second method comprises: before generating the second visible light image, adjusting a white light pulse width of the white light illumination light to achieve the adjusted white light exposure of the second visible light image; and wherein generating the second visible light image comprises: calculating the first ratio for the first visible light image; and applying one or more image processing techniques to the second image data, wherein the one or more image processing techniques are configured based at least in part on the calculated first ratio such that the second white light contribution and the second excitation contribution define the second ratio.

In some aspects of the second method, illuminating the tissue with the white light illumination light comprises illuminating with pulsed white light illumination.

In some aspects of the second method, illuminating the tissue with the excitation light comprises illuminating with constant excitation light during periods when the white light illumination is on and during periods when the white light illumination is off.

In some aspects of the second method, the excitation light is within a wavelength range of 405-415 nm.

In some aspects, a second system for white light and fluorescence imaging using visible light excitation is provided, the second system comprising: a white light illumination light that illuminates a tissue and an excitation light that illuminates the tissue, wherein the excitation light comprises light within a wavelength range of 380-490 nm; and an image sensor communicatively coupled to one or more processors, wherein the one or more processors execute instructions stored in memory to cause the system to: generate a first visible light image of the tissue based on first image data captured by the image sensor, wherein: a blue channel of the first visible light image includes a first white light contribution from reflected white light illumination light and a first excitation contribution from reflected excitation light, and the first white light contribution and the first excitation contribution define a first ratio; and after generating the first visible light image, generating a second visible light image of the tissue based on second image data captured at the image sensor, wherein: a white light exposure of the second visible light image is less than or equal to 90% of a white light exposure of the first image or is greater than or equal to 110% of the white light exposure of the first image, a blue channel of the second visible light image includes a second white light contribution from the reflected white light illumination light and a second excitation contribution from reflected excitation light, and the second white light contribution and the second excitation contribution define a second ratio that is greater than or equal to 95% of the first ratio and less than or equal to 105% of the first ratio.

In some aspects, a second non-transitory computer-readable medium storing instructions for white light and fluorescence imaging using visible light excitation is provided, wherein the instructions, when executed by one or more processors of a system comprising a white light illumination light, an excitation light, and an image sensor, cause the system to: illuminate a tissue with the white light illumination light and with the excitation light, wherein the excitation light comprises light within a wavelength range of 380-490 nm; generate a first visible light image of the tissue based on first image data captured at the image sensor, wherein: a blue channel of the first visible light image includes a first white light contribution from reflected white light illumination light and a first excitation contribution from reflected excitation light, and the first white light contribution and the first excitation contribution define a first ratio; and after generating the first visible light image, generate a second visible light image of the tissue based on second image data captured at the image sensor, wherein: a white light exposure of the second visible light image is less than or equal to 90% of a white light exposure of the first image or is greater than or equal to 110% of the white light exposure of the first image, a blue channel of the second visible light image includes a second white light contribution from the reflected white light illumination light and a second excitation contribution from reflected excitation light, and the second white light contribution and the second excitation contribution define a second ratio that is greater than or equal to 95% of the first ratio and less than or equal to 105% of the first ratio.

In some aspects, a third method for fluorescence imaging is provided, the third method comprising: illuminating a tissue region with an excitation light, wherein the tissue region comprises tissue and a target fluorophore and wherein the excitation light comprises light within a wavelength range of 380-490 nm; capturing first image data of the tissue region at one or more image sensors, wherein the first image data comprises red channel data and blue channel data; and generating a corrected fluorescence image, based on the first image data, wherein generating the fluorescence image comprises subtracting a second component based on the blue channel data from a first component based on the red channel data.

In some aspects, the third method comprises, before subtracting the second component from the first component, applying one or more preprocessing operations to one or both of the red channel data and the blue channel data.

In some aspects of the third method, capturing the first image data comprises capturing via a red channel of the one or more image sensors using a first effective gain and capturing via a blue channel of the one or more image sensors using a second effective gain that is less than the first effective gain.

In some aspects of the third method, the first effective gain is greater than or equal to 25× the second effective gain.

In some aspects of the third method: the first image data comprises green channel data; and the second component is further based on the green channel data.

In some aspects, the third method comprises, before subtracting the second sub-component from the first sub-component, applying one or more preprocessing operations to the green channel data.

In some aspects of the third method, capturing the first image data comprises capturing via a green channel of the one or more image sensors using a third effective gain that is less than the first effective gain.

In some aspects of the third method, the first effective gain is greater than or equal to 10× the first effective gain.

In some aspects of the third method, the second effective gain is equal to the third effective gain.

In some aspects of the third method, the third effective is greater than the second effective gain.

In some aspects of the third method: the steps of illuminating the tissue region with the excitation light and of capturing the first image data are performed during a fluorescence-frame time period; and the third method comprises: during a white-light-frame time period different from the fluorescence frame time-period: illuminating the tissue region with white-light illumination; capturing second image data of the tissue region at the one or more image sensors, wherein the second image data comprises red channel data, green channel data, and blue channel data; and generating a white-light image based on the second image data; and generating a color overlay image based on the corrected fluorescence image and based on the white-light image.

In some aspects of the third method, the one or more image sensors comprises a Bayer array image sensor.

In some aspects of the third method, the one or more image sensors comprises an RGB prism camera.

In some aspects, a third system, for fluorescence imaging is provided, the third system comprising: an excitation light within a wavelength range of 380-490 nm that illuminates a tissue region, wherein the tissue region comprises tissue and a target fluorophore; and one or more image sensors communicatively coupled to one or more processors, wherein the one or more processors execute instructions stored in memory to cause the system to: capture first image data of the tissue region at one or more image sensors, wherein the first image data comprises red channel data and blue channel data; and generate a corrected fluorescence image, based on the first image data, wherein generating the fluorescence image comprises subtracting a second component based on the blue channel data from a first component based on the red channel data.

In some aspects, a third non-transitory computer-readable medium, is provided, the third non-transitory computer-readable medium storing instructions for fluorescence imaging, wherein the instructions, when executed by one or more processors of a system comprising an excitation light within a wavelength range of 380-490 nm, and one or more image sensors, cause the system to: capture first image data of a tissue region at one or more image sensors, wherein the first image data comprises red channel data and blue channel data; and generate a corrected fluorescence image, based on the first image data, wherein generating the fluorescence image comprises subtracting a second component based on the blue channel data from a first component based on the red channel data.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described, by way of example only, with reference to the accompanying drawings, in which:

FIG. 1 shows an imaging system 100 configured for fluorescence imaging in the visible band, according to some aspects.

FIG. 2A shows absorption and emission spectra for Pp-IX, along with spectra for red, green, and blue color channels, according to some aspects.

FIG. 2B shows absorption and emission spectra for fluorescein, along with autofluorescence emission spectra for various types of biological tissue.

FIG. 3 shows a flowchart depicting method 300 for generating a corrected fluorescence image, according to some aspects.

FIGS. 4A-4C show flowcharts depicting method 400 for adjusting white light exposure and maintaining white balance, according to some aspects.

FIG. 5A shows flowcharts depicting method 500 for generating a corrected fluorescence image, according to some aspects.

FIG. 5B shows a graphical representation of one implementation of method 500, according to some aspects.

FIG. 5C shows a graphical representation of another implementation of method 500, according to some aspects.

FIG. 6A shows flowcharts depicting method 600 for generating a corrected fluorescence image with a color image overlay, according to some aspects.

FIG. 6B shows a graphical representation of one implementation of method 600, according to some aspects.

FIG. 7A shows flowcharts depicting method 700 for generating a corrected fluorescence image using a Bayer array sensor, according to some aspects.

FIG. 7B shows a graphical representation of one implementation of method 700, according to some aspects.

FIG. 8A shows flowcharts depicting method 800 for generating a corrected fluorescence image with a color image overlay using a Bayer array sensor, according to some aspects.

FIG. 8B shows a graphical representation of one implementation of method 800, according to some aspects.

FIG. 9 shows an example of images generated using the techniques described herein, according to some aspects.

FIG. 10 shows an example of images generated using the techniques described herein, according to some aspects.

FIG. 11 shows a computer, according to some aspects.

### DETAILED DESCRIPTION

It will be appreciated that any of the variations, aspects, features, and options described in view of the systems apply equally to the methods and vice versa. It will also be clear that any one or more of the above variations, aspects, features, and options can be combined.

### Exemplary System

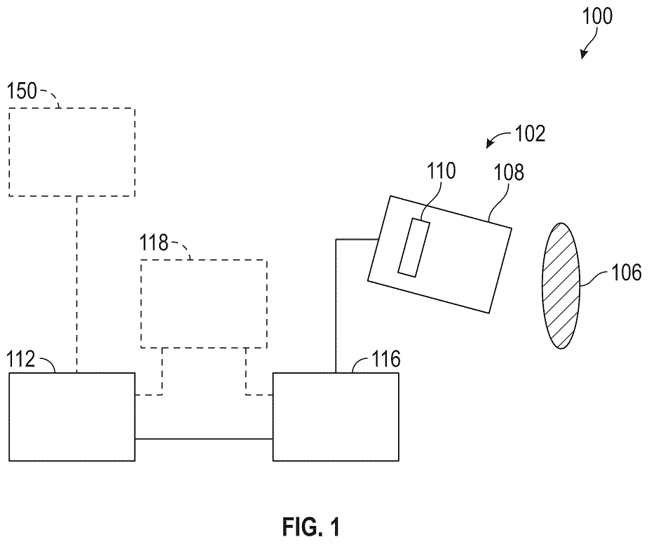

FIG. 1 illustrates an exemplary imaging system 100, according to examples of the disclosure. As described herein, system 100 may be configured for fluorescence imaging in the visible band, particularly by using a fluorescence agent (e.g., a fluorescent dye) that excites in the UV-blue range and that emits in the visible range.

As shown in FIG. 1, system 100 includes an imaging device 102 for imaging tissue 106. In some aspects, imaging device 102 may be an imaging device for insertion into a surgical cavity for imaging tissue within the surgical cavity during a medical procedure (e.g., an endoscopic imaging device), or imaging device 102 may be an imaging device for imaging outside a surgical cavity (e.g., an open-field imaging device). Imaging device 102 may include a camera head 108 that includes one or more imaging sensors 110. Light reflected and/or emitted (such as fluorescence light emitted by fluorescing targets that are excited by fluorescence excitation illumination light) from the tissue 106 is received by the camera head 108, where it is directed onto the one or more imaging sensors 110. (In endoscopic systems, the reflected and/or emitted light may be received by a distal tip of the endoscope and guided through the endoscope to the one or more imaging sensors 110.) The disclosure and the systems and methods described herein can be implemented using any suitable imaging device configured to image a patient before surgery, during surgery, after surgery, or in any other suitable medical setting.

The one or more imaging sensors 110 generate pixel data that can be transmitted to a camera control unit 116 that is communicatively connected to the camera head 108. The camera control unit 116 generates one or more images from the pixel data. The one or more images may be generated as a video feed that shows the tissue being viewed by the endoscopic imaging device 102 at any given moment in time. In one or more examples, the generated one or more images can be transmitted to an image processing unit 112 for further image processing, storage, display, and/or routing to one or more remote computing systems 150, such as a cloud computing system. The one or more images can be transmitted to one or more displays 118, from the camera control unit 116 and/or the image processing unit 112, for visualization by medical personnel, such as by a surgeon for visualizing the tissue during a surgical procedure on a patient.

Imaging device 102 may include one or more light sources, each of which may include one or more lasers, diodes, LEDs, and/or any other suitable light source type. In the example of FIG. 1, system 100 includes light source 160. In endoscopic systems, light source 160 may be optically coupled to the endoscope such that light from light source 160 is delivered (e.g., via a light cable or light cord) down the endoscope. In endoscopic and non-endoscopic systems, the light from light source 160 may be incident on tissue 106 to be imaged. Light source 160 may be controlled by camera control unit 116, which may synchronize functions of light source 160 with functions of other components of system 100. Additionally or alternatively, light source 160 may be controlled by one or more processors disposed elsewhere in system 100.

In some aspects, system 100 may be used in fluorescence imaging applications in which a exogeneous fluorescence agent (e.g., a fluorescent dye) is delivered to tissue 106 and subsequently excited by excitation light within an excitation wavelength range of the fluorescence agent. After excitation, the fluorescence agent may then emit fluorescence emission light in an emission wavelength range of the fluorescence agent. As fluorescence emission light is emitted, images of tissue 106 may be captured by system 100, where the regions of fluorescence emission light in the captured images may help to resolve anatomical features of interest.

As described above, certain fluorescence agents have excitation wavelength ranges in the UV, violet, and/or blue ranges. At these wavelength ranges, the UV, violet, and/or blue excitation light may induce autofluorescence in tissue 106, because many tissues and proteins show autofluorescence when illuminated with these wavelength ranges (e.g., at wavelengths of less than about 550 nm). Autofluorescence induced in tissue 106 may emit autofluorescence emission light primarily in the blue-green range, for example, at wavelengths of about 425-575 nm. This background autofluorescence may make it difficult to resolve regions of the tissue where the exogenous fluorophore to be imaged (e.g., the imaging agent) is actually present, because the autofluorescence contribution and fluorescence agent contribution in a visible light image captured by system 100 may both be present.

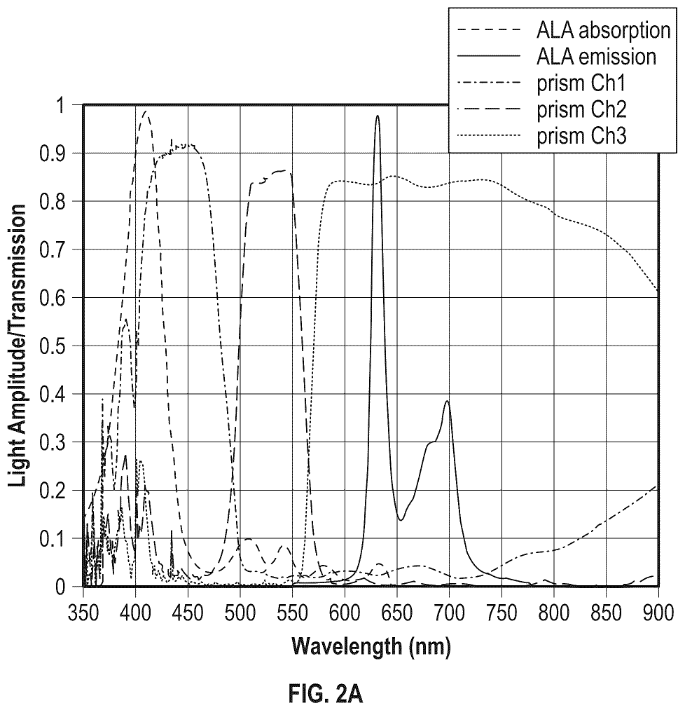

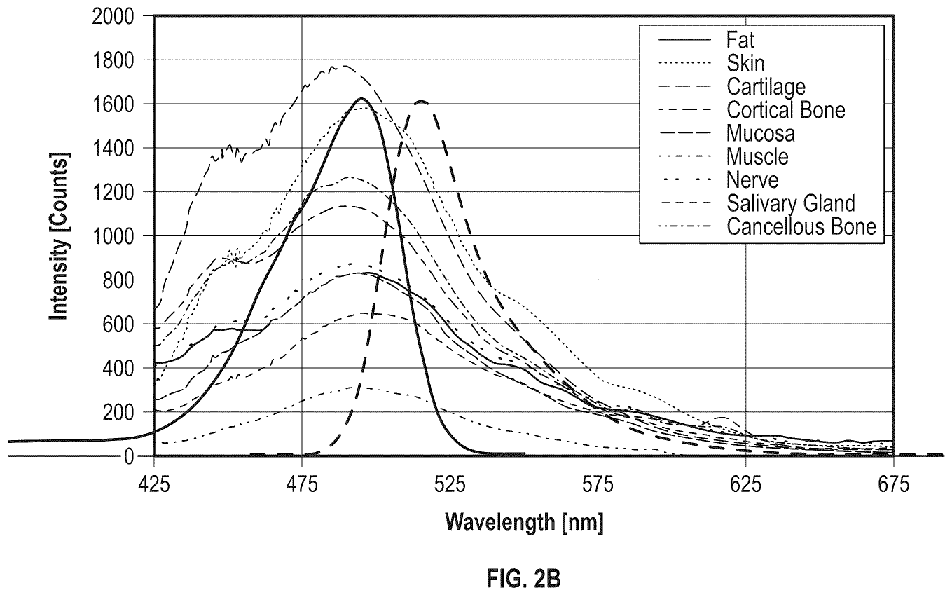

For example, FIG. 2A shows absorption and emission spectra for the 5-ala fluorophore (Pp-IX), which is one fluorescence agent that excites in the UV, violet, and/or blue range and fluoresces in the visible range. As shown in FIG. 2, Pp-IX absorbs primarily from about 375-425 nm (with non-zero absorption to a lesser extent in tails of the absorption curve below 375 nm and above 425 nm), with an absorption peak at about 405 nm. The effect of excitation light at around 405 nm on the captured image may be reduced by using a long-pass filter that blocks most of the excitation light from reaching sensors 110; however, there is a trade-off between how much excitation light of 405 nm can be blocked using a filter while still allowing sufficient blue light to be captured for a good color image to be generated. As further shown in FIG. 2A, Pp-IX emits (fluoresces) primarily between about 620 nm and 720 nm (with non-zero emission to a lesser extent in tails of the emission curve below 620 nm and above 720 nm), with a primary emission peak at about 630 nm and a secondary emission peak at about 695 nm.

Another example is shown in FIG. 2B, which shows absorption and emission spectra for fluorescein, which is another fluorescence agent that excites in the UV, violet, and/or blue range and fluoresces in the visible range. As shown in FIG. 2B, fluorescein has an absorption peak around 490 nm (as shown by the bold curve on the left in the diagram) and an emission peak around 520 nm (as shown by the bold curve on the right in the diagram).

FIG. 2B further shows autofluorescence emission spectra for various kinds of human tissue, including fat, skin, cartilage, cortical bone, mucosa, muscle, nerve, salivary gland, and cancellous bone. As shown, the autofluorescence emission ranges for these various types of tissue range primarily from 425 to 575 nm, with emission peaks centered mostly around 500 nm.

As described herein, the need to effectively resolve fluorescence emission from an exogenous imaging agent from autofluorescence emission of various tissue types may be achieved by leveraging the spectral separation between the primary emission peak of the fluorescence signal and the primary emission peak of the autofluorescence. For example, in FIG. 2B, it is shown that a primary peak of fluorescence signal for fluorescein is around 520 nm, while the primary peak of autofluorescence from tissue is around 500 nm. Taking the Pp-IX fluorescence emission band shown in FIG. 2A, the primary fluorescence emission peak is around 630 nm, which provides even more separation from a primary peak of autofluorescence from tissue of around 500 nm.

Because the different emission peaks—the fluorescence agent emission peak and the autofluorescence emission peak—are spectrally separated from one another, or the respective emission bands may have different relative emission signal portions captured by different color channels of the one or more imaging sensors 110, despite some overlap between the fluorescence agent emission band and the autofluorescence emission band. If the entire bands were themselves spectrally separated, then capturing the different bands using different sensors or using filters would be trivially simple. However, due to the overlap of the bands, more complex solutions are needed as explained herein. Because the different bands have different spectral weights, information may be effectively extracted from the different bands using the techniques described herein. Image processing techniques that selectively combine the different color channels, e.g., by weighted addition and/or by weighted subtraction, may then be used to generate a corrected fluorescence image that effectively resolves the different fluorescence contributions and effectively visualizes the regions of the tissue at which the fluorescence agent is present.

FIG. 2A shows examples of spectra for different color channels of a visible light image. As shown in FIG. 2A, the blue color channel may span from about 375-475 nm, the green color channel may span from about 475 to 575 nm, and the red color channel may span from about 575 nm upwards towards the infrared range. Incoming light may be separated into two or more different color channels using one or more prisms (e.g., Philips prims) and/or using one or more filters provided as an integrated part of the one or more imaging sensors 110 (e.g., a Bayer filter), and/or by one or more filters external to the one or more imaging sensors 110. Any suitable means for separating incoming light into separate color channels may be used.

As shown in FIG. 2A (and referencing the autofluorescence spectra shown in FIG. 2B), Pp-IX fluorescence emission may be captured primarily by a red color channel, while autofluorescence emission may be captured by the blue and/or green color channels. As shown in FIG. 2B (and referencing the color channel spectra shown in FIG. 2A), fluorescein emission may be captured primarily by a red color channel, while autofluorescence emission may be captured by the blue and/or green color channels. As shown in FIG. 2B (and referencing the color channel spectra shown in FIG. 2A), the autofluorescence peaks are in the blue/green color channel range, but the overall autofluorescence response is very wide and overlaps the fluorescence response in the 600-700 nm red color channel range.

By applying excitation light at the correct wavelengths, capturing the different signal bands (the fluorescence agent emission band and the tissue autofluorescence emission band) with different color channels, and applying appropriate image processing, the signals from the two different bands can be effectively resolved from one another, allowing an image clearly showing the fluorescence agent in the tissue to be generated. Described below are techniques for applying said excitation of tissue containing fluorescence agent in the UV/violet/blue range, image capture in the visible light range, and image processing to generate a corrected fluorescence image that allows for effective visualization of fluorescence agent regions as distinct from autofluorescence regions of tissue. Exemplary methods are described in further detail below.

### Exemplary Methods for Generating a Corrected Fluorescence Image

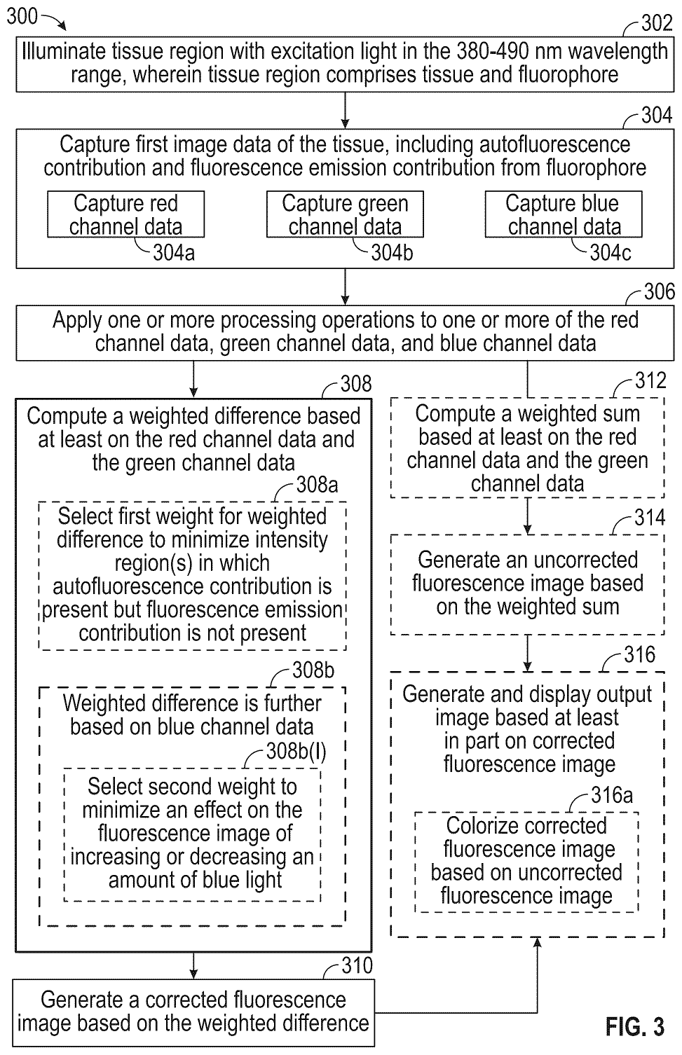

FIG. 3 shows a method 300 for generating a corrected fluorescence image, in accordance with some aspects. Method 300 may be performed by a system for medical fluorescence imaging, such as system 100 as shown in FIG. 1.

At block 302, the system may illuminate a tissue region with an excitation light in the 380-490 nm wavelength range, wherein the tissue region comprises tissue and a fluorophore. As described above, the fluorophore may be an exogenous fluorescence agent that has been administered into the tissue before illumination, such as Pp-IX or fluorescein. While illumination light in the 380-490 nm range is referred to herein, other wavelength ranges may be used, for example, greater than or equal to 350, 360, 370, 380, 390, 400, or 410 nm and less than or equal to 460, 470, 480, 490, 500, 510, 520, or 530 nm.

The illumination light in the 380-490 nm wavelength range may be selected and configured in accordance with an absorption peak of the fluorescence agent in the tissue, for example, such that the illumination light is centered on or around the absorption peak. The illumination light wavelength may also be selected and or configured in a manner that seeks to minimize autofluorescence from the tissue, for example, by choosing wavelengths of illumination light that are shifted away from wavelengths that will induce tissue autofluorescence, to the extent that the illumination wavelength light can still effectively excite the fluorophore.

The illumination light may be such that it excites the exogeneous fluorophore and causes fluorescence from the fluorophore while also causing autofluorescence from the tissue.

Optionally, the system may illuminate the target tissue with one or more additional wavelengths and/or one or more additional light sources, for example, by using an LED light source in the blue range to provide some visible light for background illumination (as opposed to for fluorescence excitation).

At block 304, the system may capture first image data of the tissue, wherein the captured image data includes a fluorescence emission contribution from the exogeneous fluorophore and also includes an autofluorescence contribution from the tissue. The image data of the tissue may be captured by one or more imaging sensors of the system.

In some aspects, the system may use a violet LED that illuminates at the 400-420 nm range (e.g., at a range centered on and/or with an intensity peak at 410 nm). Optionally, the system may use a long-pass filter, or other filter such as a notch filter, to block this excitation light (or most of it) from reaching imaging sensors of the system.

As noted at blocks 304a, 304b, and 304c, the captured first image data may include red channel data, blue channel data, and/or green channel data. Light contributing to the red channel, blue channel, and green channel may be separated and/or filtered using a Philips prism and/or a color filter array sensor (such as a Bayer sensor) and may be incident on one or more imaging sensor(s) such as imaging sensor(s) 110, which may include a CMOS (global or rolling shutter), a CCD, or any other suitable imaging sensor type. In some aspects, the system may expose all three color channels at the same time, in order to prevent motion artifacts.

The fluorescence emission contribution of the first image data may contribute to one or more of the channels. The autofluorescence contribution of the first image data may contribute to one or more of the channels. In some examples, such as in some use cases where the fluorophore is Pp-IX, the fluorescence emission contribution may be primarily in the red channel while the autofluorescence contribution may be primarily in the blue and green channels.

At block 306, the system may apply one or more processing operations to one or more of the red channel data, green channel data, and blue channel data. For example, the system may apply one or more pre-processing operations and/or filtering operations. Processing operations may be used to remove noise and/or to correct for one or more differences such as pixel offset, different surface response, or smoothing. Pre-processing operations may include filtering a second component before using it to compute a weighted difference, as described below.

At blocks 308-310, the system may use the color channel data (e.g., optionally by using the color channel data after pre-processing) to generate a corrected fluorescence image based on the color channel data. In some aspects, the red channel may include significant contribution from the fluorescence emission contribution, but may also include some contribution from autofluorescence of the tissue. Thus, as described in further detail below, the system may subtract at least some amount of the green channel data from the red channel data in order to generate a corrected fluorescence image. As described below, the system may generate a corrected fluorescence image based on a weighted difference computed based on at least two terms, wherein each of the terms are based on color-channel values (e.g., green, red, or blue, respectively).

At block 308, the system may compute a weighted difference based at least on the red channel data and the green channel data. A component of the difference based on the red channel data may be weighted or unweighted, and a component of the difference based on the green channel data may be weighted or unweighted. The weighted difference may be used to generate a corrected fluorescence image and may be denoted as:

$$F ( x , y ) = R ( x , y ) - α G ( x , y ) \qquad ( 1 )$$

where F represents corrected fluorescence image data at a given x-y pixel, R represents red-channel image data at the given pixel, G represents green-channel image data at the pixel, and a represents a constant that can be tuned for the particular imaging system and imaging application being used, calibrated for a system, and/or dynamically adjusted depending on the detailed implementation.

In some aspects, tuning may include imaging some samples known to include only the imaging agent, and other samples known to include only the tissue type intending for examination (e.g., a representative source of autofluorescence), and then adjusting the factors to attain desired separation. Autofluorescence comes from many different molecules, and so the resulting factors may be a compromise between eliminating all possible autofluorescence at the expense of hiding some of the drug fluorescence, versus letting a small amount of autofluorescence through in order to ensure that faint drug levels are still visible. Choice between these two options may depend on the particular surgery or other application. Generally, a should be tuned such that in absence of the fluorophore the image is mostly black.

At block 308a, optionally, computing the weighted difference comprises selecting a first weight for the weighted difference to minimize one or more intensity regions in which autofluorescence contribution is present but fluorescence emission contribution is not present (or is minimally present, e.g., below a predefined or dynamically determined threshold level). In the example formulation at equation (1) above, the first weight may be a. In some aspects, a may be tuned such that, in the absence of the fluorophore, the corrected fluorescence image is mostly black.

At block 308b, optionally, computing the weighted difference is further based on the blue channel data (in addition to the red channel data and the green channel data). In some aspects, because the green image channel may potentially be polluted by blue light leaking (e.g., because the color filter isn't very narrow), depending on the illumination and filter configuration, some amount of blue may be subtracted from green to remove this leakage. For example, the corrected fluorescence image F may be calculated as:

$$F ( x , y ) = R ( x , y ) - α ( G ( x , y ) - β B ( x , y ) ) \qquad ( 2 )$$

where B represents blue-channel image data at the pixel and β is a constant that can be tuned for the particular imaging system and imaging application being used, calibrated for a system, and/or dynamically adjusted depending on the detailed implementation. Tuning may include imaging subjects that are known to contain only the target fluorophore, and other subjects that are known to contain only autofluorescence, and subjects containing both, and then adjusting the factors to attain sufficient separation of the two signals.

At block 308b(i), optionally, the second weight is selected to minimize the effect on the corrected fluorescence image of increasing or decreasing the amount of blue light. In the example formulation at equation (1) above, the first weight may be α. In some aspects, β should be tuned such that any increase/decrease of the blue light does not impact the generated corrected fluorescence image, or such that the effect on the generated corrected fluorescence image is minimized or mitigated (for example, by being maintained below a predefined or dynamically determined threshold level).

At block 310, the system may generate the corrected fluorescence image based on the weighted difference computed at block 308. For example, the corrected fluorescence image may be generated as (or based on) the weighted difference shown by equation (1) or the weighted difference shown by equation (2).

In some aspects, the generated corrected fluorescence image may be displayed, stored, transmitted to one or more other systems, used to create one or more visualizations (e.g., by being combined into a composite image with one or more other images), subject to additional image processing operations, and/or used to trigger one or more automated system functionalities (e.g., by automatically triggering one or more medical device or surgical device functionalities in accordance with whether the generated corrected fluorescence image meets one or more predefined or dynamically determined criteria).

At block 316, the system may generate and display an output image based at least in part on the corrected fluorescence image. For example, in some aspects, the corrected fluorescence image may be displayed as an output image, for example, by being displayed on a display without being subject to further image processing.

Turning now to blocks 312-314 and 316a, optional steps for generating an uncorrected fluorescence image and for colorizing the corrected fluorescence image based on the uncorrected fluorescence image are described.

At block 312, which may follow from block 306 and may proceed in parallel to blocks 308-310, optionally, the system may compute a weighted sum based at least on the red channel data and the green channel data. A component of the sum based on the red channel data may be weighted or unweighted, and a component of the sum based on the green channel data may be weighted or unweighted.

In some aspects, the weighted sum may be based on a first term based on the red channel data and a second term based on the green channel data. One or both of the terms may be weighted. In some aspects, weighting of one or both of the terms for the weighted sum may be the same weighting that is used in equation (1) or equation (2) above. In some aspects, weighting of one or both of the terms for the weighted sum may be a different weighting than is used in equation (1) or equation (2) above.

At block 310, optionally, the system may generate an uncorrected fluorescence image based on the weighted sum computed at block 312. For example, the uncorrected fluorescence image may be generated as (or based on) the weighted sum.

At block 316, optionally, generating and displaying the output image may comprise colorizing the corrected fluorescence image based on the uncorrected fluorescence image. In this manner, regions at which the exogeneous fluorescence agent is present in the tissue may be highlighted using colorization, rather than relying solely on luminance in a visible light image for a fluorescence agent that emits in the visible spectrum.

In some aspects, the weighted difference and/or the corrected fluorescence image generated based thereon may be used to generate an image, overlay, or other visualization for a drug (e.g., exogeneous fluorescence agent) or autofluorescence, and that image, overlay, or other visualization may, for example, be scaled between neutral and a specific color. In some aspects, the weighted difference may be used to calculate, for each pixel, the ratio of drug to autofluorescence, and that ratio may be applied to a chroma difference (e.g., by mapping that ratio to the C-R axis such that 100:0 is cyan, 50:50 is grey and 0:100 is red). In each of those cases, in some aspects, the subtraction noise is mostly in the chromas, which can be aggressively smoothed without much loss of perceived image quality, as long as the luminance component is not overly noisy. Using this approach, the user may be presented with an output visualization that visualizes the likelihood of a particular fluorescence region being the imaging agent versus autofluorescence.

### Exemplary Methods for Adjusting White Light Exposure and Maintaining White Balance

In some aspects, illumination of tissue for fluorescence imaging may involve illuminating the tissue with both white light illumination and with fluorescence excitation light in the 380-490 nm wavelength range. For example, in some applications in which a fluorescence agent that excites in the UV/violet/blue wavelength range is used, fluorescence excitation light in the 380-490 nm wavelength range used to excite the fluorescence agent may be provided simultaneously with—or in rapid succession in a pulsed manner with—white light used to generate a white light image.

In some aspects, illuminating tissue with both fluorescence excitation light in the UV/violet/blue wavelength range and with white light may be performed over a period of time such that multiple images of the same tissue are captured, for example, multiple images of tissue in a time series such as a video feed. In some aspects, white light exposure for one or more images in the time series may be adjusted over time with respect to one or more prior images in the time series.

However, when white light exposure changes over time between different images in the time series, it is desirable that a white balance of the different images should be held constant (or as close to constant as is reasonably possible). For example, if a white light pulse width is adjusted, the amount of visible blue light in the captured image may change, because the UV-based excitation light (which may in some aspects remain on rather than being pulsed) may be detected in the blue channel of the captured images. More specifically, the blue channel of an image may comprise a white light contribution from reflected white-light illumination light and may include an excitation light contribution from reflected excitation light. The white light contribution and the excitation light contribution may define a ratio, and it is desirable that that ratio should be held constant between different images captured in a time series of images of tissue, even as the white light exposure may change between those different images.

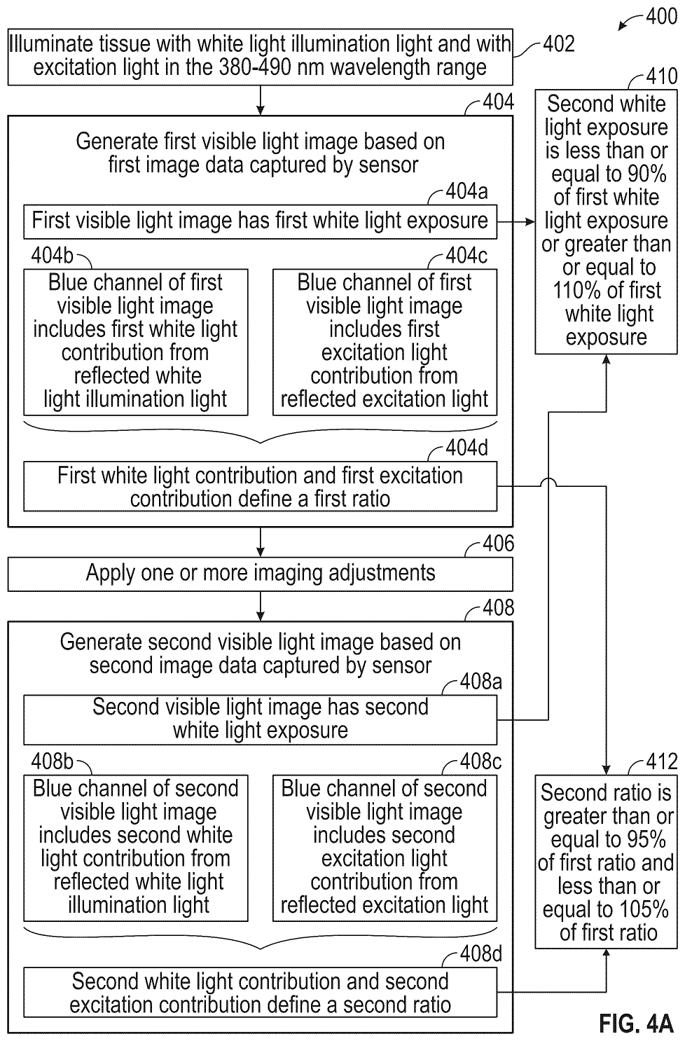

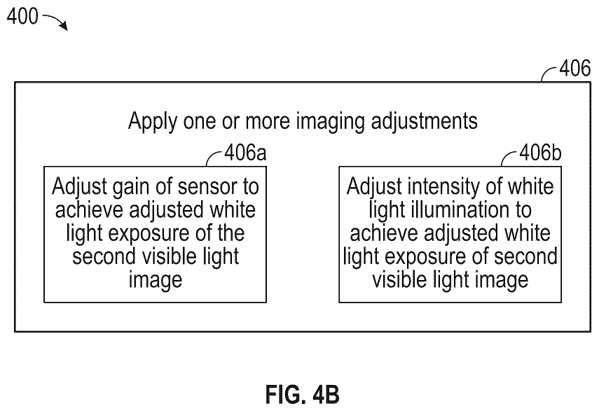

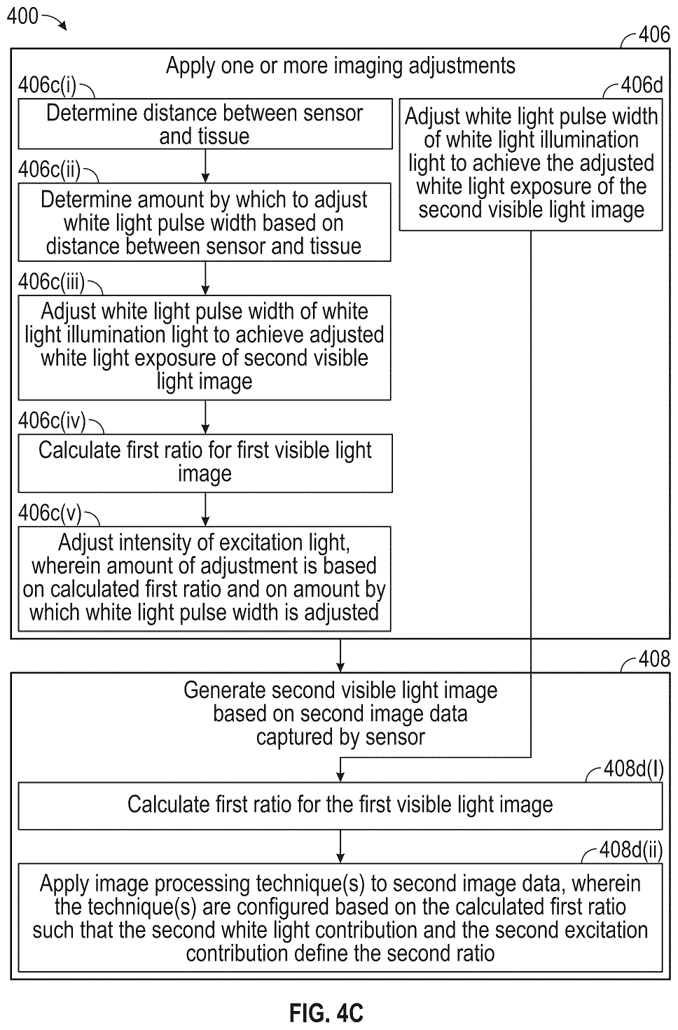

Described below in detail with reference to FIGS. 4A-4C are imaging techniques in which different images have varying white light exposures but for which the above-described ratio of contributions to the blue channel are held relatively stable. Several exemplary imaging adjustment techniques that contribute to these imaging techniques are described.

FIGS. 4A-4C show a method 400 for generating a corrected fluorescence image, in accordance with some aspects. Method 400 may be performed by a system for medical fluorescence imaging, such as system 100 as shown in FIG. 1.

At block 402, the system may illuminate a tissue region with an excitation light in the 380-490 nm wavelength range and with white light illumination. In some aspects, the tissue may comprise a fluorophore (e.g., an exogenous fluorescence agent) that has been administered into the tissue before illumination, such as Pp-IX or fluorescein, that excites in the 380-490 nm wavelength range. Illumination of the tissue may share any one or more characteristics in common with the illumination techniques described above with reference to block 302 in method 300.

In some aspects, the system may use multiple different light sources to provide the excitation light and the white light. For example, the system may include four LED light sources for illumination, including a fluorescence excitation LED in the 380-490 nm wavelength range. The fluorescence excitation LED may be a violet LED that emits light centered at 410 nm. The four light sources may further include blue (e.g., about 465 nm), red (e.g., about 620 nm), and green (e.g., about 530 nm) LEDs for providing the white light illumination.

In some aspects, the illumination light may be provided using a pulsed illumination scheme in which one or more wavelength ranges of the illumination light are temporally pulsed (e.g., turned on and off). The precise timing scheme for any one or more wavelength ranges (e.g., for any one or more LEDs) used to provide the illumination light may be adjusted according to system settings and/or user input, and/or dynamically according to feedback detected by one or more system sensors (including imaging sensors of the system).

In some aspects, a timing scheme may be used in which the excitation light source (e.g., a violet LED) remains on constantly throughout the entire illumination and image capture process. Meanwhile the white light illumination source(s), such as red, green, and blue LEDs, may be pulsed. The pulse width used for the white light illumination source(s) may be adjusted over time to be widened or narrowed according to needs of specific system settings, a specific application, and/or a specific environment. In some aspects, a timing scheme may be used such that certain captured frames contain only fluorescence, alternating with frames containing fluorescence as well as white light, where the white light signal generally overwhelms the fluorescence

At block 404, the system may generate a first visible light image based on first image data captured by an imaging sensor. In the example of system 100, visible light may be captured by one or more imaging sensors 110, and system 100 may generate a visible light image based on the captured image data.

During image capture, the one or more imaging sensors 110 may be set to a specific gain that may be adjusted (e.g., increased or decreased) according to needs of specific system settings, a specific application, and/or a specific environment.

The captured first image data used to generate the first visible light image may include red channel data, blue channel data, and/or green channel data. Light contributing to the red channel, blue channel, and green channel may be separated and/or filtered using a Philips prism and/or Bayer sensor and may be incident on the one or more imaging sensors used by the system, such as imaging sensor(s) 110, which may be a CMOS (global or rolling shutter), a CCD, or any other suitable imaging sensor type. In some aspects, the system may expose all three color channels at the same time, in order to prevent motion artifacts.

Optionally, a long-pass filter (or other filter such as a notch filter), such as a 458 nm long-pass filter, may be used during image capture to block some (e.g., most) of the excitation light from the reaching the one or more imaging sensors.

The first visible light image generated at block 404 may have various characteristics, as described for example at blocks 404a-404d.

As noted at block 404a, the first visible light image generated based on the first image data captured by the one or more sensors may have a first white light exposure value (which may be quantified in arbitrary units).

As noted at blocks 404b and 404c, the blue channel of the first visible light image may include a first white light contribution from reflected white light illumination light, as well as a first excitation light contribution from reflected excitation light.

As noted at block 404d, a first ratio may be defined by (a) the first white light contribution to the blue channel of the first white light image and (b) the first excitation light contribution to the blue channel of the first white light image. White light contribution and excitation light contribution may be defined by respective amounts by which the different light sources contribute to image sensor values (e.g., intensity values as measured by the image sensor).

After generating the first image based on first image data captured by the one or more imaging sensors of the system, the method may proceed to block 406, where one or more imaging adjustments are applied by the system. The one or more imaging adjustments may include one or more adjustments to illumination settings of the system, one or more adjustments to image sensor and/or image data capture settings, and/or one or more adjustments to image data processing settings and/or image post-processing settings.

Adjustments that may be applied at block 406 are described in greater detail below with reference to FIGS. 4B and 4C.

After applying the one or more imaging adjustments at block 406, the method may proceed to block 408, at which the system may generate a second visible light image based on second data captured by the one or more imaging sensors of the system. The second image data may be captured by the same set of one or more imaging sensors that was used at block 404 to capture the first image data. The capture of the second image data may be performed at a time subsequent to the capture of the first image data, for example, by capturing data for a subsequent frame in a video feed (though the second image and the first image need not represent immediately adjacent frames in a video feed, as there may be one or more intermediate frames).

The capture of the second image data and the generation of the second visible light image at block 408 may share any one or more characteristics in common with the capture of the first image data and the generation of the first visible light image, as described above with reference to block 404. The second visible light image may have analogous characteristics, as described above with respect to blocks 404a-d, including a second white light (block 408a), a second white light contribution to the blue channel from reflected white light illumination light (block 408b), a second excitation light contribution to the blue channel from reflected excitation light (block 308c), and a second ratio (block 408d) defined by (a) the second white light contribution to the blue channel of the second white light image and (b) the second excitation light contribution to the blue channel of the second white light image.

As noted above, there may be certain imaging applications in which it is desirable for certain characteristics of the second image to vary from the corresponding characteristics of the first image, while certain other characteristics of the same second image should remain the same (or nearly the same) as those corresponding characteristics of the first image. One example of this situation is in imaging applications in which it is desirable to adjust the white light exposure of the second image with respect to the first image, for example, by increasing or decreasing the white light exposure of the second image with respect to the first image (e.g., as a part of applying the one or more imaging adjustments at block 406). One example of a scenario in which white light exposure of the second image with respect to the first image may be adjusted is a scenario in which adjustments are made to the imaging scene; relevant factors may include working distance, tissue color, imaging angle, cavity size, and endoscope size.

As noted above, one problem introduced by adjusting white light exposure levels between different images is that the adjustment can inadvertently lead to changes in white balance. For example, in scenarios in which white light illumination light is being pulsed with UV/violet/blue excitation light in the 380-490 nm wavelength range, reflected excitation light may be partially visible in the blue channel of the one or more image sensors of the system. Thus, if the white light pulse width is adjusted between different images, the relative amount of visible blue light in the blue channel of the one or more image sensors may change between the different images. For example, if the white light pulse width is decreased and no other imaging adjustments are made, and a UV/violet/blue excitation light remains constantly on, then the ratio of contribution to the blue channel from reflected excitation light to the contribution to the blue channel from reflected white light may increase, and the overall intensity of blue light in the image relative to the red and green color channel intensities may increase. Conversely, if the white light pulse width is increased and no other imaging adjustments are made, and a UV/violet/blue excitation light remains constantly on, then the ratio of contribution to the blue channel from reflected excitation light to the contribution to the blue channel from reflected white light may decrease, and the overall intensity of blue light in the image relative to the red and green color channel intensities may decrease.

To account for this phenomenon, specific techniques may be used to apply the one or more imaging adjustments at block 406 and/or to capture image data for the second image and generate the second visible light image at block 408. Various examples of such techniques are explained with reference to FIGS. 4B and 4C.

In FIG. 4B, block 406a shows a first optional imaging adjustment technique in which applying the one or more imaging adjustments comprises adjusting a gain of one or more image sensors to achieve an adjusted white light exposure of the second visible light image. Sensor gain may be adjusted to increase or decrease white light exposure of the second visible light image, as desired, without the need to change a white light illumination pulse width. By avoiding adjusting the white light illumination pulse width, the relative amounts of contribution to the blue channel from UV/violet/blue excitation light and from white light illumination light may remain the same for the second image as they were for the first image, and the color balance of the two images may therefore be the same. However, it should be noted that this first optional technique may reduce the dynamic range of the system.

Block 406b shows a second optional imaging adjustment technique in which applying the one or more imaging adjustments comprises adjusting an intensity of white light illumination to achieve an adjusted white light exposure of the second visible light image. The amplitude of the white light pulses may be adjusted up or down, in some aspects with or without adjusting their pulse width and/or optionally in combination with one or more other imaging adjustments, for example to ensure the white light balance is not changed. This technique may require a very good calibration/white balance of the light source. The amplitude of a light source may be adjusted by adjusting the current flowing through an LED of the light source. The light output from the LED may not be linear to the current. At the same time the light output relative to the pulse width may be mostly constant (ignoring rise/fall times). Thus, the current to light output may need to be understood well to adjust the amplitude of a UV/blue/violet excitation light source in line with the pulse width of white light.

In FIG. 4C, blocks 406c(i)-406c(v) show a third optional imaging adjustment technique in which applying the one or more imaging adjustments comprises a series of steps, as described below.

At block 406c(i), the system may determine a distance between the system's one or more imaging sensors and the tissue to be imaged. Optionally, in addition to or rather than determining a distance between the one or more imaging sensors and the tissue, the system may determine a distance between the tissue and a frontmost imaging tip of the system. One or more proximity sensors may be used to determine said distance.

At block 406c(ii), the system may determine an amount by which to adjust a white light pulse width of the white light illumination light, wherein the adjustment amount is determined based at least in part on the distance determined at block 406c(i). Less illumination may be needed when the distance to the tissue is lowered, so the illumination intensity may accordingly be lowered. Conversely, higher illumination intensity may be provided when the distance is increased.

At block 406v(iii), the system may adjust the white light pulse width of the white light illumination light in accordance with the amount determined at block 406c(ii).

At block 406c(iv), the system may calculate the first ratio for the first image. That is, the system may calculate a ratio defined by (a) the first white light contribution to the blue channel of the first white light image and (b) the first excitation light contribution to the blue channel of the first white light image.

At block 406c(v), the system may then adjust an intensity of the excitation light (e.g., the 380-490 nm wavelength range excitation light), wherein the amount of adjustment of the intensity of the excitation light is determined based at least in part on the calculated first ratio and on the amount by which the while light pulse width was (or is to be) adjusted. The system may compute an amount by which to adjust the intensity of the excitation light in order to compensate for a change in white balance that would be expected to be caused by the change in white light pulse width. For example, the system may compute the first ratio and then determine an amount by which to change the excitation light intensity in order to ensure that the second ratio for the second image is the same as (or within a threshold percentage of) the first ratio.

In some aspects, a change in the white light pulse width may have a linear effect on the optical power hitting the tissue, and thus also on the white light image signal, proportional to the pulse width. The excitation light may be adjusted in the same manner, by pulsing it, and/or by some other means such as varying the current. The relationship between current and optical power may not be linear, but from the controls point of view it may be desirable to be able to adjust the optical power of the excitation light in a linear fashion in order to match what is happening with the white light illumination.

Turning now to blocks 406d, 408d(i), and 408d(ii), a fourth optional imaging adjustment technique is shown in which adjustments to illumination are made and then image processing techniques are applied to the second visible light image after the second visible light image is generated (or while the second visible light image is being generated).

At block 406d, applying the one or more imaging adjustments comprises adjusting a white light pulse width of the white light illumination light to achieve an adjusted white light exposure of the second visible light image. Image data for the second visible light image is then captured while using the adjusted white light pulse width.

At block 408d(i), generating the second visible light image based on the second image data captured by the one or more imaging sensors comprises calculating the first ratio for the first visible light image. That is, the system may calculate a ratio defined by (a) the first white light contribution to the blue channel of the first white light image and (b) the first excitation light contribution to the blue channel of the first white light image.

At block 408d(ii), generating the second visible light image based on the second image data captured by the one or more imaging sensors comprises then applying one or more image processing techniques to the captured second image data, wherein the one or more image processing techniques are configured based on the calculated first ratio such that the second white light contribution to the blue channel of the second image and the second excitation light contribution to the blue channel of the second image define the second ratio. Applying the one or more image processing techniques to the second white light image may adjust a white balance (e.g., may adjust the second ratio for the second image) such that the white balance of the second white light image is prevented from deviating (or deviating too far) from a white balance of the first white light image. This may be accomplished without otherwise adjusting the illumination intensity or sensor settings.

By applying one or more of the image adjustment techniques described in FIG. 4B or 4C (or by applying another suitable image adjustment technique at block 406), the white light exposure of the second image may be adjusted with respect to the white light exposure of the first image, while the white balance of the second image (e.g., as quantified by the second ratio) may be held constant or close to constant with respect to the white balance of the first image (e.g., as quantified by the first ratio).

For example, as shown at block 410, the second white light exposure may be less than or equal to 90% of the first white light exposure or greater than or equal to 110% of the first white light exposure. In some aspects, the second white light exposure may be (a) less than or equal to 1%, 5%, 10%, 25%, 50%, 75%, 90%, or 95% of the first white light exposure or (b) greater than or equal to 105%, 110%, 125%, 150%, 175%, 200%, 500%, or 1,000% of the first white light exposure.

Meanwhile, as shown at block 412, the second ratio may be greater than or equal to 95% of the first ratio and less than or equal to 105% of the first ratio. (It should be understood that each ratio may be converted to a number value, e.g., 2:1=1; 1:1=1; 1:2=0.5. Those independent number values may then be used to calculate the comparison percentages referenced at block 412.) In some aspects, the second ratio may be (a) greater than or equal to 50%, 75%, 90%, 95%, 99%, 99.9%, or 99.99% of the first ratio and (b) less than or equal to 100.01%, 100.1%, 101%, 105%, 110%, 125%, or 150% of the first ratio.

In some aspects of method 400, a filter (e.g., a clip filter) may be used to mitigate or remove the effects on the captured images of autofluorescence of the tissue.

In some aspects, method 400 may be combined (in whole or in part) and/or applied in parallel with method 300, for example, to generate an adjusted fluorescence image as described in method 300 while also maintaining white light balance across different images in a time series as described in method 400. In some aspects, method 400 may be combined (in whole or in part) and/or applied in parallel with any other method or technique described herein, to generate fluorescence images and/or color-overlay images while also maintaining white light balance across different images in a time series.

Exemplary Methods for Generating a Corrected Fluorescence Image Correcting for Leakage of UV, Violet, or Blue Excitation Light into the Fluorescence (e.g., Red) Channel

In some aspects, filterless fluorescence imaging with illumination in the UV, violet, and/or blue range and imaging in the white light range is enabled by illuminating tissue with UV, violet, and/or blue illumination, collecting fluorescence imaging data using a red channel (e.g., a red sensor), and collecting background imaging data using one or both of a blue channel and a green channel. In some aspects, background imaging data may be collected using a blue channel only (and, e.g., the green channel may be ignored); in some aspects, background imaging data may be collected using both the blue and green channel.

In some aspects, a Blue-Green offset (BG offset) may be applied. In some aspects, more BG offset may be applied to allow for large signal difference between blue and red. The offset may be chosen such that the blue and/or green channel have a balanced exposure (highlights are not blown out and detail is retained in the shadows) and the red (fluorescence) channel is at the user desired intensity to see a feature of interest.

In some aspects using an RGB prism implementation, the three sensors may be running at different gains and/or different exposure periods. An effective difference in gain (combined gain and exposure) may determine the fluorescence sensitivity. In cases in which no excitation-blocking filter is used, it may be desirable for that difference to be greater by about 30 dB or 32 times. For example, in implementations that use a filter, a user may be able to adjust offset from +0 dB to +27 dB; however, in implementations that do not use a filter, an adjustable range for offset may be restricted to about +30 dB to about +57 dB, combined gain and exposure length difference.

In some aspects, different exposure lengths per frame may be used for the red channel, green channel, and/or blue channel. In some aspects, the exposure of the blue and/or green channels may be about 1/10 or less per frame than the exposure of the red channel. In some aspects, the exposure of the blue and/or green channels may be about 1/25 or less per frame than the exposure of the red channel. In some aspects, the exposure of the blue channel may be about 1/25 or less per frame than the exposure of the red channel, and the exposure of the green channel may be about 1/10 or less per frame than the exposure of the red channel.

A difference may then be calculated based on at least the red channel data and the blue channel data (and optionally the green channel data) to generate a corrected fluorescence image based on the calculated difference. In some aspects, the system may estimate leakage into the red channel by multiplying the blue image by a factor that includes the known relative sensitivity of the red and blue channels to the excitation band, and the relative gains (combined gain and exposure factor) of the two sensors (or, in the case of Bayer, the two images used for fluor and correction). That estimated leakage image may then be subtracted from the red image to get net fluorescence. The net fluorescence may be used for the corrected fluorescence image. The corrected fluorescence image may thus provide a corrected image, based on the background imaging data, that corrects for leakage of the UV, violet, and/or blue illumination light into the visible light sensor range.

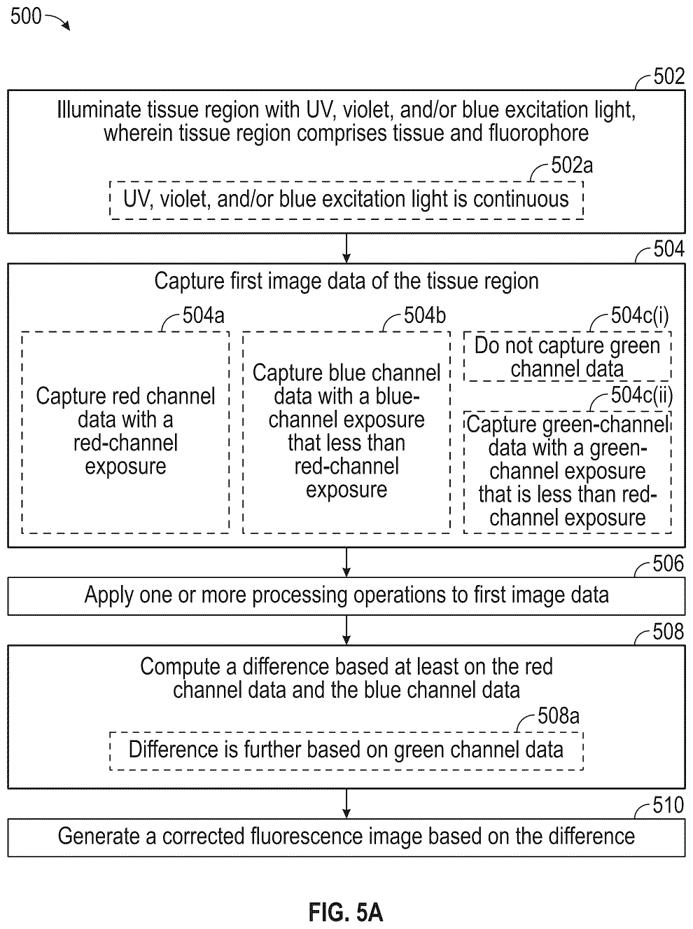

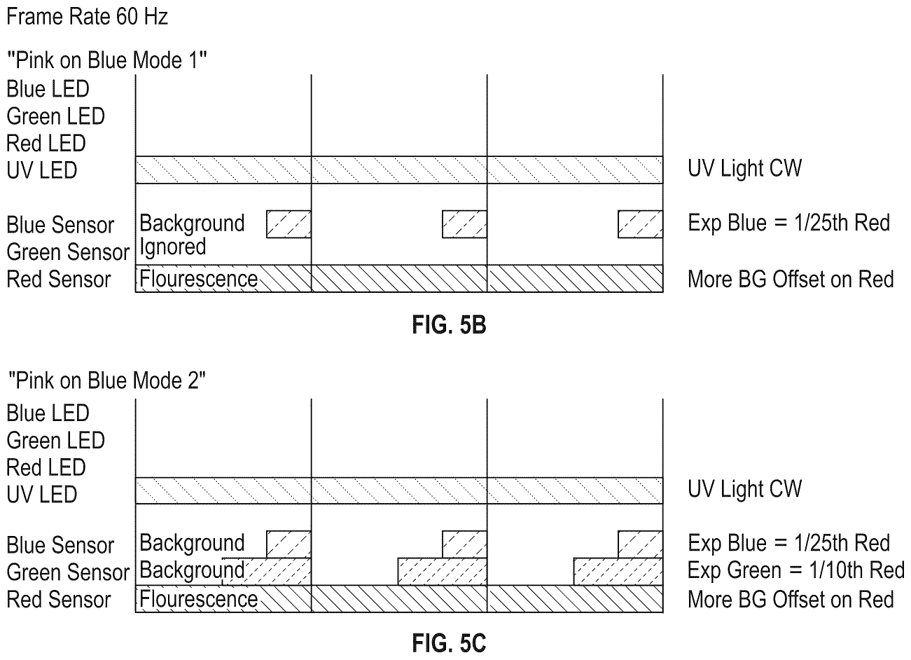

FIG. 5A shows flowcharts depicting method 500 for generating a corrected fluorescence image, according to some aspects. Method 500 may be performed by a system for medical fluorescence imaging, such as system 100 as shown in FIG. 1.

At block 502, the system may illuminate a tissue region with an excitation light in the UV, violet, and/or blue range. The illumination may be in the 380-490 nm wavelength range. In some aspects, the tissue may comprise a fluorophore (e.g., an exogenous fluorescence agent) that has been administered into the tissue before illumination, such as Pp-IX or fluorescein, that excites in the 380-490 nm wavelength range. Illumination of the tissue may share any one or more characteristics in common with the illumination techniques described above with reference to block 302 in method 300.

At block 502a, the UV, violet, and/or blue light excitation is continuous. The illumination may be provided as continuous-wave illumination, rather than being pulsed and/or otherwise time-varied.

At block 504, the system may capture first image data of the tissue region. The image data of the tissue may be captured by one or more imaging sensors of the system.

As noted at blocks 504a, 504b, and 504c, the captured first image data may include red channel data, blue channel data, and/or green channel data. Light contributing to the red channel, blue channel, and green channel may be separated and/or filtered using a Philips prism and/or a color filter array sensor (such as a Bayer sensor) and may be incident on one or more imaging sensor(s) such as imaging sensor(s) 110, which may include a CMOS (global or rolling shutter), a CCD, or any other suitable imaging sensor type. In some aspects, the system may expose all three color channels at the same time, in order to prevent motion artifacts.

At block 504a, the system captures red channel data with a red-channel exposure. The red-channel exposure may have a corresponding exposure period. In some aspects, the red-channel exposure may be continuous. In some aspects, the red-channel may last for an entire frame of the imaging scheme.

At block 504b, the system captures blue channel data with a blue-channel exposure that is less than the red-channel exposure. In some aspects, the blue-channel exposure (e.g., an exposure period) may be less than or equal to 50%, 25%, 10%, 5%, or 1% of the red-channel exposure. In some aspects, the blue-channel exposure (e.g., an exposure period) may be greater than or equal to 50%, 25%, 10%, 5%, or 1% of the red-channel exposure. In some aspects, the blue-channel exposure may be about 1/25 the red-channel exposure.

Capturing green-channel data is optional in method 500.

At block 504c(i), the system does not capture green channel data.

At block 504c(ii), as an alternative to 504b(i), the system captures green channel data with a green-channel exposure that is less than the red-channel exposure In some aspects, the green-channel exposure is greater than the blue-channel exposure. In some aspects, the green-channel is greater than or equal to 2× the blue-channel exposure. In some aspects, the green-channel exposure is about 2.5× the blue-channel exposure. In some aspects, the green-channel exposure (e.g., an exposure period) is less than or equal to 50%, 25%, 10%, 5%, or 1% of the red-channel exposure. In some aspects, the green-channel exposure (e.g., an exposure period) is greater than or equal to 50%, 25%, 10%, 5%, or 1% of the red-channel exposure. In some aspects, the blue-channel exposure may be about 1/10 the red-channel exposure.

At block 506, the system applies one or more processing operations to the first image data. For example, the system may apply one or more pre-processing operations and/or filtering operations. Processing operations may be used to remove noise and/or to correct for one or more differences such as pixel offset, different surface response, or smoothing. Pre-processing operations may include filtering a second component before using it to compute a difference, as described below.

At block 508, the system computes a difference based at least on the red-channel data and the blue-channel data. A component of the difference based on the red-channel data may be weighted or unweighted, and a component of the difference based on the blue-channel data may be weighted or unweighted. The difference may be used to generate a corrected fluorescence image and may be denoted as:

$$F ( x , y ) = R ( x , y ) - α B ( x , y ) \qquad ( 3 )$$

where F represents corrected fluorescence image data at a given x-y pixel, R represents red-channel image data at the given pixel, B represents green-channel image data at the pixel, and a represents a constant that can be tuned for the particular imaging system and imaging application being used, calibrated for a system, and/or dynamically adjusted depending on the detailed implementation.

At block 508a, optionally, the difference is further based on the green-channel data (in addition to the red channel data and the blue channel data). For example, the corrected fluorescence image F may be calculated as:

$$F ( x , y ) = R ( x , y ) - α B ( x , y ) - β G ( x , y ) \qquad ( 4 )$$

where G represents green-channel image data at the pixel and β is a constant that can be tuned for the particular imaging system and imaging application being used, calibrated for a system, and/or dynamically adjusted depending on the detailed implementation.

At block 510, the system may generate the corrected fluorescence image based on the difference computed at block 508. For example, the corrected fluorescence image may be generated as (or based on) the weighted difference shown by equation (3) or the weighted difference shown by equation (4).

In some aspects, the generated corrected fluorescence image may be displayed, stored, transmitted to one or more other systems, used to create one or more visualizations (e.g., by being combined into a composite image with one or more other images), subject to additional image processing operations, and/or used to trigger one or more automated system functionalities (e.g., by automatically triggering one or more medical device or surgical device functionalities in accordance with whether the generated corrected fluorescence image meets one or more predefined or dynamically determined criteria).

FIG. 5B shows a graphical representation of one implementation of method 500, according to some aspects. As shown, the implementation in FIG. 5B uses continuous-wave illumination in the UV/violet/blue range, and for collection ignores the green channel and collects blue-channel data at an exposure that is 1/25 the exposure used for the red-channel data.

FIG. 5C shows a graphical representation of another implementation of method 500, according to some aspects. As shown, the implementation in FIG. 5C uses continuous-wave illumination in the UV/violet/blue range, collects green-channel data at an exposure that is 1/10 the exposure used for the red-channel data, and collects blue-channel data at an exposure that is 1/25 the exposure used for the red-channel data.

In some aspects, the above-described techniques, of collecting fluorescence imaging data using a red channel and background imaging data using a blue channel and optionally a green channel, may be applied in a time-splicing schema. In these aspects, UV, violet, and/or blue illumination may be pulsed alternately with pulsed white-light illumination. The collection using the red channel for fluorescence imaging data and the blue and/or green channels for background imaging data (optionally, with different exposures for the different sensors) may be performed during UV, violet, and/or blue illumination pulses, while collection of white-light color-image data may be performed using all three channels of the white-light sensor (optionally, with equal exposures to one another) during pulses of the white-light illumination. This may allow for both a corrected fluorescence image and a color image to be generated, optionally for overlaid display with one another.

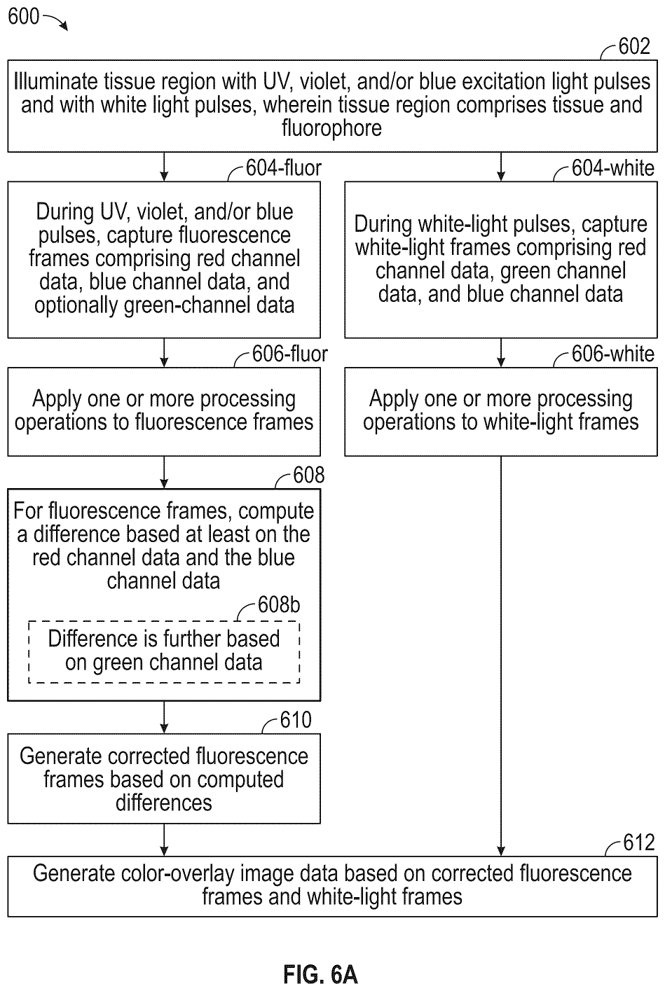

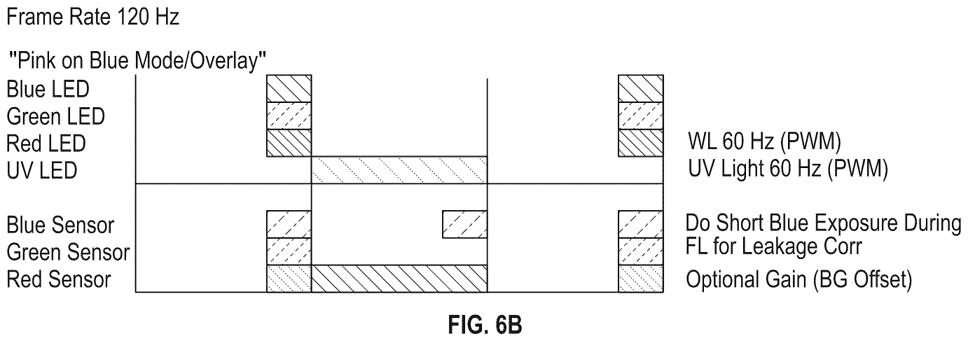

FIG. 6A shows flowcharts depicting method 600 for generating a corrected fluorescence image with a color image overlay, according to some aspects. Method 600 may be performed by a system for medical fluorescence imaging, such as system 100 as shown in FIG. 1.

At block 602, the system may illuminate a tissue region with UV, violet, and/or blue excitation light pulses and with white light pulses, wherein the tissue region comprises tissue and fluorophore. The UV/violet/blue pulses may be configured to excite the fluorophore, while the white light pulses may be configured to enable generating white-light images, for example for creation of a fluorescence and color-image overlay.

The fluorescence excitation illumination may be in the 380-490 nm wavelength range. Fluorescence excitation pulses (e.g., the UV/violet/blue light pulses) may share any one or more characteristics in common with the illumination techniques described above with reference to block 502 in method 300.

The white light illumination may be in the 400-700 nm wavelength range.

Fluorescence excitation pulses and white light pulses may alternate with one another back and forth in a pattern of two pulses, or they may be interleaved with other illumination pulses in a pattern of more than two pulses. Fluorescence excitation pulses and white light pulses may be of equal lengths of time to one another, or they may differ in length (e.g., the fluorescence excitation pulses may be longer than the white light pulses, or the white light pulses may be longer than the fluorescence excitation pulses). Fluorescence excitation pulses and white light pulses may directly abut one another in time, or they may be spaced from one another by a period during which no illumination is provided.

Illumination pulses may match the full duration of the corresponding sensor acquisition frame, or may be shorter if less light is needed. Global shutter sensors may adjust their exposure length by draining the charge mid-frame, so the active period may ends at the end of the frame period, and its length may be adjusted by timing that drain pulse. Thus, the system may be configured to align the illumination pulse with that active period. Fluorescence may be relatively weaker weak, such that it may be advantageous to capture a full-frame exposure of fluorescence light to read it with the least gain, while reflectance may be relatively brighter, so it may be advantageous to shorten that white-light pulse to avoid saturation even at minimum gain. Thus, in some aspects, the UV pulse may be full length, while the white light pulse may be shorter and may be aligned to the end of that frame period.

At blocks 604-fluor through 610, which may follow from block 602, the system may capture fluorescence frames during UV/violet/blue light excitation pulses, process said captured fluorescence excitation data, and generate corrected fluorescence images based on said captured and optionally processed fluorescence excitation data. Blocks 604-fluor through 610 may share any one or more characteristics in common with corresponding blocks in blocks 504-510 as described above with reference to FIG. 5A. (For example, the exposure of a red sensor during fluorescence frame capture may be longer than the exposure of a blue sensor, and may be longer than an optional exposure of a green sensor.)

At block 604-white, which may follow from block 602, the system may capture white-light frames during white-light excitation pulses. The captured white-light frames may comprise red-channel data, green-channel data, and blue-channel data. Exposure for a red channel, green channel, and blue channel may be equal to one another during white-light frame capture. At the same imaging distance, the system may use different exposure lengths for white light versus for fluorescence. In some aspects, exposure lengths for fluorescence may be greater than exposure lengths for white-light imaging. The relationship between exposure length for fluorescence imaging and for white-light imaging may be user-adjusted in order to change the fluorescence sensitivity.

At block 606-white, the system may apply one or more processing operations to the white-light frames. Processing operations applied at block 606-white may share any one or more characteristics in common with those applied at block 506 as described above with reference to FIG. 5A.

At block 612, which may follow from blocks 610 and 606-white, the system may generate color-overlay image data based on the corrected fluorescence frames and based on the white-light frames.

FIG. 6B shows a graphical representation of one implementation of method 600, according to some aspects. In the example of FIG. 6B, white light frames and fluorescence frames are alternated with one another, with the white-light illumination provided in short pulses at the end of the frame and the UV/violet/blue illumination provided in longer pulses spanning the full length of the frame. In the example of FIG. 6B, white light data is collected during white-light frames by exposing the blue, green, and red sensors equally (for a short exposure length) during the white-light illumination. In the example of FIG. 6B, fluorescence frames are collected during the UV/violet/blue illumination by exposing the red sensor for a relatively longer exposure length and exposing the blue sensor for a relatively shorter exposure length.

In some aspects, a Bayer array sensor may be used. This may avoid the need for use of a prism, which may allow for more compact devices.

In some aspects using a Bayer array sensor, continuous UV, violet, and/or blue illumination may be applied, fluorescence imaging data may be collected in fluorescence frames using only red pixels of the Bayer array sensor and using a first exposure for the red pixels, and background frames may be collected using only the blue (and optionally the green) pixels, and using exposure(s) that is/are less than the first exposure. It may be advantageous to use a full-frame exposure to get a sufficiently bright fluorescence image on the red channel, but that same exposure length may saturate the blue pixels, since they are getting the full excitation power. If the blue pixels are saturated, then an accurate correction cannot be calculated, so the background frame may be taken at a shorter exposure, which may then be multiplied up according to the ratio of exposure lengths. Corrected background frames may be generated by adjusting color balance. Corrected fluorescence images may then be generated, which correct for UV, violet, and/or blue illumination light leakage into the visible light sensor range, based on the fluorescence frames and on the corrected background frames.

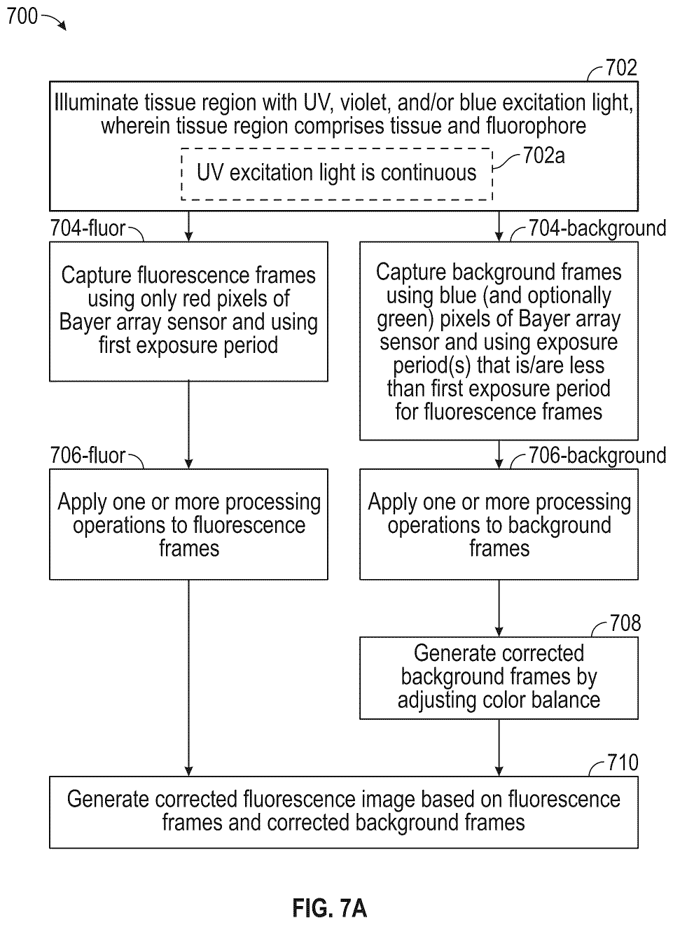

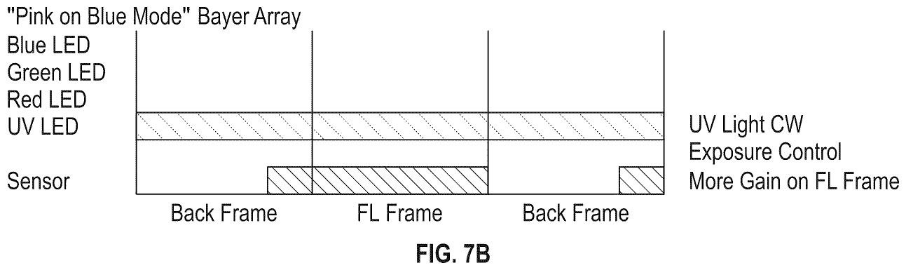

FIG. 7A shows flowcharts depicting method 700 for generating a corrected fluorescence image using a Bayer array sensor, according to some aspects. Method 700 may be performed by a system for medical fluorescence imaging, such as system 100 as shown in FIG. 1.

At block 702, the system may illuminate a tissue region with an excitation light in the UV, violet, and/or blue range. The illumination may be in the 380-490 nm wavelength range. In some aspects, the tissue may comprise a fluorophore (e.g., an exogenous fluorescence agent) that has been administered into the tissue before illumination, such as Pp-IX or fluorescein, that excites in the 380-490 nm wavelength range. Illumination of the tissue may share any one or more characteristics in common with the illumination techniques described above with reference to block 302 in method 300.

At block 702a, the UV, violet, and/or blue light excitation is continuous. The illumination may be provided as continuous-wave illumination, rather than being pulsed and/or otherwise time-varied.

At block 704-fluor, the system may capture fluorescence frames using only red pixels of a Bayer array sensor and using a first exposure period. At block 704-background, the system may capture background frames using blue (and optionally green) pixels of the Bayer array sensor and using one or more exposure periods that are less than the first exposure period used for the red pixels at block 704-fluor. In some aspects, the exposure periods used for the red, blue, and optionally green pixels at blocks 704-fluor and 704-background may have the same relationships (e.g., relative exposure lengths) to one another as described above with reference to FIG. 5A for the red, blue, and optionally green channels in method 500.

Capturing red-channel, blue-channel, and optionally-green channel data at blocks 704-fluor and 704-background may share any one or more characteristics in common with capturing image data as described above with respect to block 504 of FIG. 5A.

At block 706-fluor, the system may apply one or more processing operations to the fluorescence frames. At block 706-background, the system may apply one or more processing operations to the background frames. Processing operations applied at block 706-fluor and/or 706-background may share any one or more characteristics in common with those applied at block 506 as described above with reference to FIG. 5A.

At block 708, the system may generate corrected background frame by adjusting color balance of the captured and/or processed background frames. In some aspects, adjusting the color balance may be performed by, for example, scaling the contribution from one or more of the pixel colors with respect to one or more of the other pixel colors. In some aspects, the system may scale up the green contribution. In some aspects, the system may scale up the green contribution by a factor of 2. In some aspects, if using green data and blue data from the “background” frame, then that data may be scaled to provide a desired balance of green to blue.

At block 710, the system may generate a corrected fluorescence image based on the fluorescence frames and the corrected background frames. The corrected fluorescence image may be generated by calculating a difference based on the fluorescence frames and the corrected background frames, for example as described above with reference to blocks 508-510 and/or 608-610.

FIG. 7B shows a graphical representation of one implementation of method 700, according to some aspects. As shown, in the example of FIG. 7B, continuous UV/violet/blue illumination is used, and background frames are collected in alternation with fluorescence frames. Fluorescence frames are collected with a longer exposure than is used for background frames. There is a temporal spacing between fluorescence light collection and background light collection, while there is no temporal spacing between background light collection and fluorescence light collection.

In some aspects using a Bayer array sensor, UV, violet, and/or blue illumination may be pulsed with white-light illumination. During periods of UV, violet, and/or blue illumination, fluorescence frames may be collected using red pixels of the Bayer array sensor at a first exposure. During periods of white light illumination, optionally, correction frames may be collected using blue pixels of the Bayer away sensor and using a second exposure shorter than the first exposure. During periods of white light illumination, optionally, white-light frames may be collected using red, green, and blue pixels of the Bayer array sensor and using a third exposure shorter than the first exposure. Optionally, the white light frames may be used for automatic gain control. Optionally, the white light frames may be used to spoof G/B background. Optionally, corrected fluorescence frames may be generated by performing background subtraction based on the fluorescence frames and the background frames and/or based on spoofed G/B background from the white-light frames. Optionally, color overlay image data may be generated based on the corrected fluorescence frames and the white-light frames.

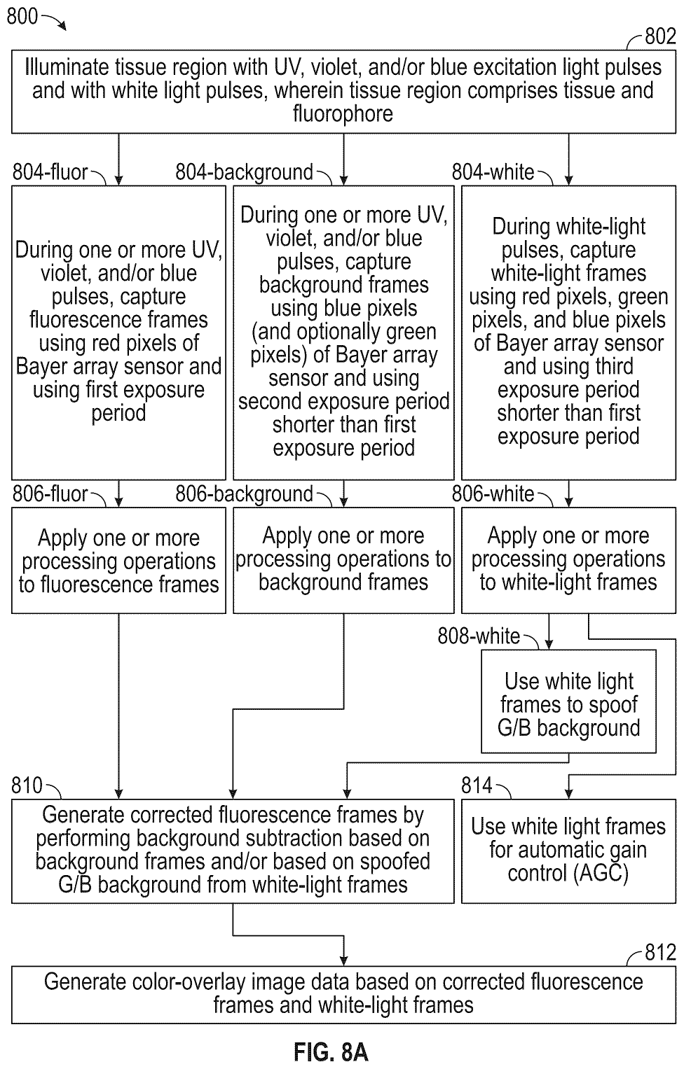

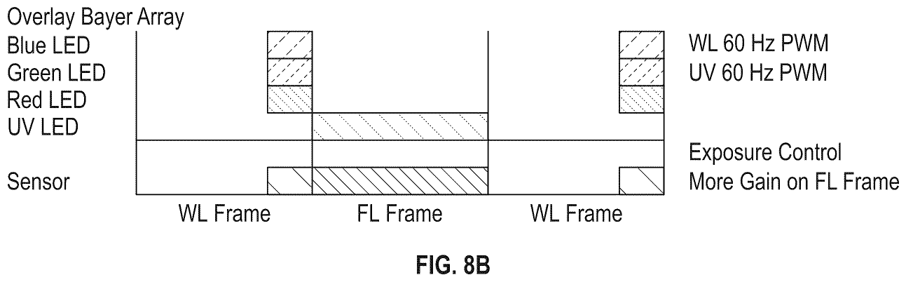

FIG. 8A shows flowcharts depicting method 800 for generating a corrected fluorescence image with a color image overlay using a Bayer array sensor, according to some aspects. Method 800 may be performed by a system for medical fluorescence imaging, such as system 100 as shown in FIG. 1.

At block 802, the system may illuminate a tissue region with UV, violet, and/or blue excitation light pulses and with white light pulses, wherein the tissue region comprises tissue and fluorophore. The UV/violet/blue pulses may be configured to excite the fluorophore, while the white light pulses may be configured to enable generating white-light images, for example for creation of a fluorescence and color-image overlay. Illumination at block 802 may share any one or more characteristics in common with illumination at block 602 as described above with reference to method 600 at FIG. 6A.

At block 804-fluor, the system may, during one or more UV, violet, and/or blue light illumination pulses, capture fluorescence frames using red pixels of a Bayer array sensor and using a first exposure period. Block 804-fluor may share any one or more characteristics in common with block 704-fluor of method 700 described above with reference to FIG. 7A.

At block 804-background, the system may, during one or more UV, violet, and/or blue light illumination pulses, capture background frames using blue pixels (and optionally green pixels) of the Bayer array sensor and using a second exposure period shorter than first exposure period. Block 804-background may share any one or more characteristics in common with block 704-background of method 700 described above with reference to FIG. 7A.

At block 804-white, the system may, during one or more white-light pulses, capture white-light frames using red pixels, green pixels, and blue pixels of the Bayer array sensor and using a third exposure period that is shorter than first exposure period. The third exposure period may be the same as or may be different from the second exposure period.

The capture of background frames may be optional, such that in some aspects fluorescence frames and white-light frames are captured but background frames are not captured. The capture of white-light frames may be optional, such that in some aspects fluorescence frames and background frames are captured but white-light frames are not captured.

The timing schema used for illumination may be to alternate between excitation pulses and white-light pulses. The excitation pulses and white-light pulses may be the same length as one another, or may be different lengths from one another. The pulses may directly temporally abut one another or may be offset from one another.

The timing schema used for capture of frames may be to alternate between fluorescence and background frames, in aspects that do not used white-light frames. The timing schema used for capture of frames may be to alternate between fluorescence and white-light frames, in aspects that do not used background frames. The timing schema used for capture of frames may be to repeatedly cycle through the three frame types in any given order. The timing schema used for capture of frames may be to capture fluorescence, then background, then fluorescence, then white-light, then to repeat the pattern of four.

In some aspects using a three-frame cycle, a short-exposure correction frame may be placed immediately before a long-exposure fluorescence frame, such that the light contributing to the correction image is provided as close in time as possible to the image that is intended to correct. For example, a frame order may be white light, correction, fluorescence (and then repeat). Shortened exposures may be provided at the end of their frame period.

At block 806-fluor, the system may apply one or more processing operations to the fluorescence frames. At block 806-background, the system may apply one or more processing operations to the background frames. At block 806-white, the system may apply one or more processing operations to the white-light frames. Processing operations applied at block 806-fluor, 806-background, and/or 806-white may share any one or more characteristics in common with those applied at block 506 as described above with reference to FIG. 5A.

At block 808, the system may use the one or more captured and/or processed white-light frames to spoof background frames, such as background frames based on blue-channel image data and green-channel image data. In some aspects, the reflected excitation light may be replaced with reflected blue from the white light image. Scaling may be used according to the ratio of illumination power between the two sources, the relative sensitivity of the image sensor at those two wavelengths, the relative transmission of the illumination and imaging paths, and/or an estimate of the relative reflectance of the subject at those wavelengths. This approach may be useful in situations in which a system always or usually runs in an overlay mode.

At block 810, the system may generate corrected fluorescence frames by performing background subtraction based on the background frames and/or based on the spoofed background from white-light frames. In some aspects, the system may calculate a difference as described above herein. In some aspects, generating corrected fluorescence frames at block 810 may share any one or more characteristics in common with block 710 described above with reference to FIG. 7A.

At block 812, the system may generate color-overlay image data based on the corrected fluorescence frames and based on the white-light frames.

At block 814, the system may use the white-light frames for automatic gain control (AGC) and/or automatic gain and exposure control. In some aspects, fluorescence imaging systems may apply automatic gain and exposure control based on a reflected white light image, and may then set the fluorescence gain and/or exposure relative to that. That approach may be preferred for overlay imaging (e.g., overlay or CSF), since it may ensure that regions with the same drug concentration look the same regardless of distance and angle. In a filterless blue approach, it may be desirable for the fluorescence gain to be set by reflected excitation light rather than by reflected white light, and when the system is not acquiring a white light image (e.g., in pink-on-blue mode), the fluorescence gain may only depend on the reflected excitation light.

FIG. 8B shows a graphical representation of one implementation of method 800, according to some aspects. AS shown, in the example of FIG. 8B, white-light frames are captured in an alternating manner with a fluorescence frame. During the white-light frames, white-light illumination using a red LED, green LED, and blue LED is provided in a short illumination period adjacent the end of the frame. During the white-light frames, the Bayer array sensor is used to capture white-light image data using red pixels, blue pixels, and green pixels during a short exposure adjacent the end of the frame and aligned with the white-light illumination period. During the fluorescence frame, a UV/violet/blue LED is illuminated for the entire frame length, and the red pixels of the Bayer array sensor are used to capture fluorescence image data.

In alternative aspects to the two-frame rotation of FIG. 8B, a shorter exposure may be used not to saturate blue pixels.

### Example #1

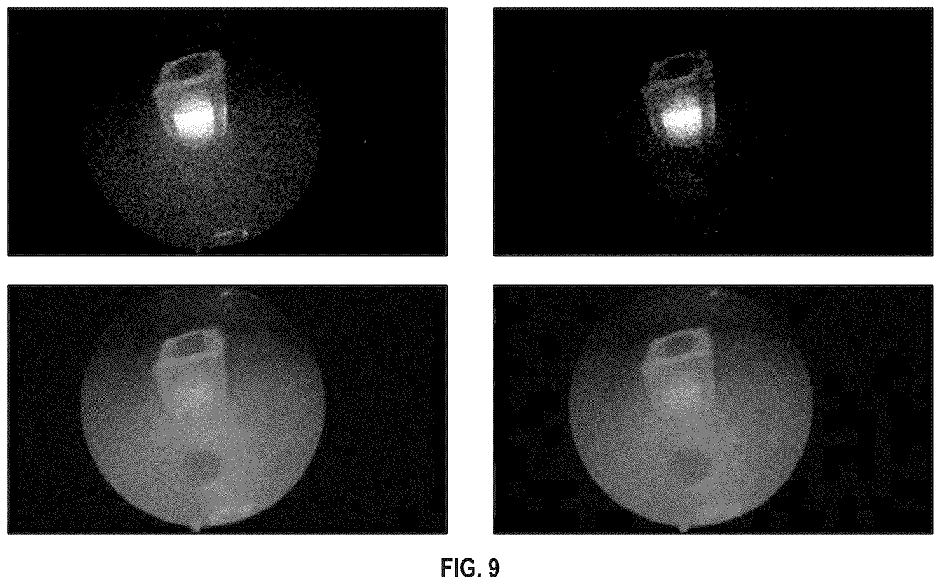

FIG. 9 shows an example in which images were generated using techniques described herein. The images in the left column show uncorrected fluorescence images (which may be red-channel images). The images in the right column show corrected fluorescence images that were generated based on a weighted difference computed from a red channel component and a green channel component. Specifically, 22% of the green channel component was subtracted from the red channel component to generate the corrected fluorescence images shown. The upper images show fluorescence on its own (which may be referred to as “contrast mode”). The lower images show a color image with fluorescence overlaid in green (which may be referred to as “overlay mode”).

### Example #2

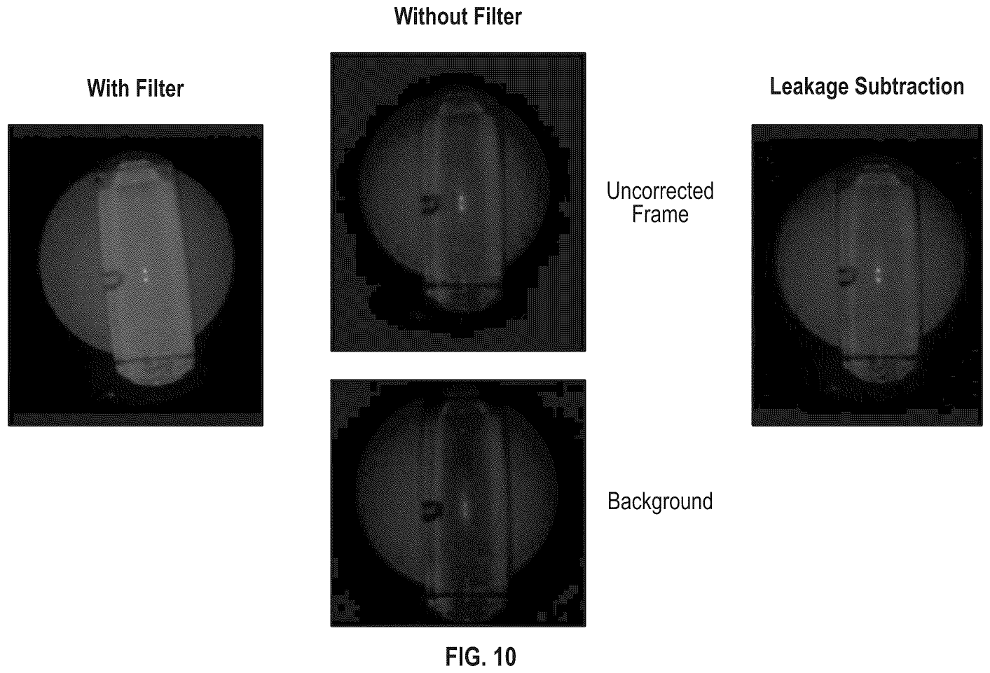

FIG. 10 shows an example in which images were generated using techniques described herein. The image on the left shows an image captured under UV/violet/blue illumination with a filter used; this is a composite image, using a filter under continuous UV illumination, such that the result is the data from the red channel for fluorescence and the blue channel for background. The image at the top center shows an image captured under UV/violet/blue illumination, using blue and red channels, without a filter used, without leakage correction applied yet. The image at the bottom center shows a background image captured under UV/violet/blue illumination using blue-channel image data. The image on the right shows a corrected fluorescence image generated by subtracting the background image (bottom center) from the uncorrected frame (top center). In this example, the following difference was used to generated the corrected fluorescence image:

$${FL}_{corr \_ raw} = {FL}_{raw} - α \frac{{gain}_{FL} * {SH}_{FL}}{{gain}_{Blue} * {SH}_{FL}} {Blue}_{raw} \qquad ( 5 )$$

where FL_{corr_raw} is the corrected fluorescence image data, FL_{raw} is the uncorrected frame data (after black level subtraction), gain_{FL} is gain of the uncorrected frame, SH_{FL} is shutter duration (e.g., exposure length) of the uncorrected frame, a is a leakage ratio that is empirically derived and may in some examples be between about 0.008 and 0.01, Blue_{raw} is the background image (after black level subtraction), gain_{Blue} is gain of the background image, and SH_{Blue} is shutter duration (e.g., exposure length) of the background image. Using this equation, the fluorescence remains at the appropriate scale for combining with white light for overlay. Gain in the Equation 5 is linear gain multiplier, not a log-scale dB measure, and shutter duration is the exposure time. Units for shutter do not matter in Equation 5, since one shutter duration is divided by another.

### Exemplary Computer

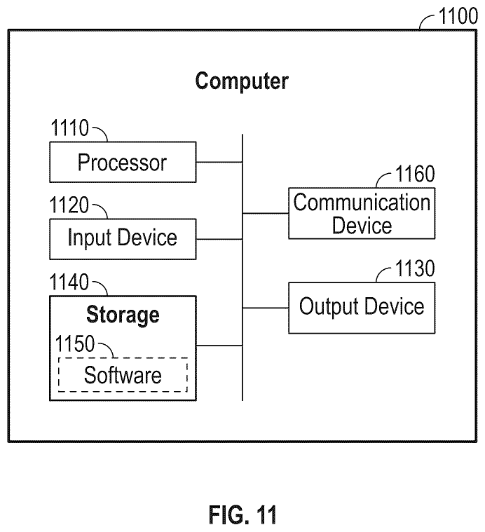

FIG. 11 illustrates an example of a computing system 1100, in accordance with some examples of the disclosure. Computing system 1100 can be a client or a server. As shown in FIG. 11, computing system 1100 can be any suitable type of processor-based system, such as a personal computer, workstation, server, handheld computing device (portable electronic device) such as a phone or tablet, or dedicated device. The computing system 1100 can include, for example, one or more of input device 1120, output device 1130, one or more processors 1110, storage 1140, and communication device 1160. Input device 1120 and output device 1130 can either be connectable or integrated with computing system 1100.

Input device 1120 can be any suitable device that provides input, such as a touch screen, keyboard or keypad, mouse, gesture recognition component of a virtual/augmented reality system, or voice-recognition device. Output device 1130 can be or include any suitable device that provides output, such as a display, touch screen, haptics device, virtual/augmented reality display, or speaker.

Storage 1140 can be any suitable device that provides storage, such as an electrical, magnetic, or optical memory including a RAM, cache, hard drive, removable storage disk, or other non-transitory computer readable medium. Communication device 1160 can include any suitable device capable of transmitting and receiving signals over a network, such as a network interface chip or device. The components of the computing system 1100 can be connected in any suitable manner, such as via a physical bus or wirelessly.

Processor(s) 1110 can be any suitable processor or combination of processors, including any of, or any combination of, a central processing unit (CPU), field programmable gate array (FPGA), and application-specific integrated circuit (ASIC). Software 1150, which can be stored in storage 1140 and executed by one or more processors 1110, can include, for example, the programming that provides the functionality or portions of the functionality of the present disclosure (e.g., as described with respect to the systems and methods, as described above).

Software 1150 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1140, that can contain or store programming for use by or in connection with an instruction execution system, apparatus, or device.

Software 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer-readable medium can include, but is not limited to, an electronic, magnetic, optical, electromagnetic, or infrared wired or wireless propagation medium.

Computing system 1100 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computing system 1100 can implement any operating system suitable for operating on the network. Software 1150 can be written in any suitable programming language, such as C, C++, Java, or Python. In various aspects, application software providing the functionality of the present disclosure can be deployed in different configurations, such as in a client/server arrangement or through a Web browser as a Web-based application or Web service, for example.

The foregoing description, for the purpose of explanation, has been described with reference to specific aspects and examples. However, the illustrative discussions above are not intended to be exhaustive or to limit the invention to the precise forms disclosed. Many modifications and variations are possible in view of the above teachings. The aspects and examples were chosen and described in order to best explain the principles of the techniques and their practical applications. Others skilled in the art are thereby enabled to best utilize the techniques and various aspects with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

For the purpose of clarity and a concise description, features are described herein as part of the same or separate examples; however, it will be appreciated that the scope of the disclosure includes examples having combinations of all or some of the features described.

### ENUMERATED EXAMPLES

The following examples are exemplary and are not intended to limit the scope of the disclosure provided herein.

Example 1. A method for fluorescence imaging, the method comprising:
illuminating a tissue region with an excitation light, wherein the tissue region comprises tissue and a target fluorophore and wherein the excitation light comprises light within a wavelength range of 380-490 nm;
capturing first image data of the tissue at an image sensor, wherein the first image comprises an autofluorescence contribution from the tissue and a fluorescence emission contribution from the target fluorophore, wherein the first image data comprises red channel data and green channel data; and
generating a corrected fluorescence image, based on the first image data, wherein generating the fluorescence image comprises subtracting a second component based on one of the green channel data or the red channel data from a first component based on the other of the green channel data or the red channel data.

Example 2. The method of example 1, wherein the second component comprises a first weight.

Example 3. The method of example 2, comprising selecting the first weight to minimize intensity of the fluorescence image in one or more regions of the fluorescence image in which the autofluorescence contribution is present but the fluorescence emission contribution is not present.

Example 4. The method of any one of examples 1-3, comprising, before subtracting the second component from the first component, applying one or more preprocessing operations to one or both of the red channel data and the green channel data.

Example 5. The method of any one of examples 1-4, wherein the second component is based on the red channel data and the first component is based on the green channel data.

Example 6. The method of any one of examples 1-5, wherein the second component is based on the green channel data and the first component is based on the red channel data.

Example 7. The method of example 6, wherein:
the first image data comprises blue channel data; and
the second component is computed by subtracting a second sub-component based on the blue channel data from a first sub-component based on one of the green channel data or the red channel data.

Example 8. The method of example 7, wherein the second sub-component comprises a second weight.

Example 9. The method of example 8, comprising selecting the second weight to minimize an effect on the fluorescence image of increasing or decreasing an amount of blue light.

Example 10. The method of any one of examples 7-9, comprising, before subtracting the second sub-component from the first sub-component, applying one or more preprocessing operations to the blue channel data.

Example 11. The method of any one of examples 1-10, wherein capturing the first image data comprises exposing a red color channel, a green color channel, and a blue color channel simultaneously.

Example 12. The method of any one of examples 1-11, comprising displaying an output image, wherein displaying the output is based at least in part on the generated corrected fluorescence image.

Example 13. The method of example 12, comprising generating the output image, wherein generating the output image comprises:
generating an uncorrected fluorescence image based on the first image data; and
colorizing the corrected fluorescence image based on the uncorrected fluorescence image, wherein the colorization distinguishes the autofluorescence contribution from the fluorescence emission contribution.

Example 14. The method of example 13, wherein generating the uncorrected fluorescence image comprises summing a fourth component based on one of the green channel data or the red channel data with a third component based on the other of the green channel data or the red channel data.

Example 15. A system for fluorescence imaging, the system comprising:
an excitation light that illuminates a tissue region with an excitation light, wherein the tissue region comprises tissue and a target fluorophore and wherein the excitation light comprises light within a wavelength range of 380-490 nm;
an image sensor that captures first image data of the tissue, wherein the first image comprises an autofluorescence contribution from the tissue and a fluorescence emission contribution from the target fluorophore, wherein the first image data comprises red channel data and green channel data; and
one or more processors, coupled to the image sensor, that execute instructions stored in memory to generate a corrected fluorescence image, based on the first image data, wherein generating the fluorescence image comprises subtracting a second component based on one of the green channel data or the red channel data from a first component based on the other of the green channel data or the red channel data.

Example 16. A non-transitory computer-readable medium storing instructions for fluorescence imaging, wherein the instructions, when executed by one or more processors of a system comprising an excitation light and an image sensor, cause the system to:
illuminate, by the excitation light, a tissue region with an excitation light, wherein the tissue region comprises tissue and a target fluorophore and wherein the excitation light comprises light within a wavelength range of 380-490 nm;
capture, by the image sensor, first image data of the tissue, wherein the first image comprises an autofluorescence contribution from the tissue and a fluorescence emission contribution from the target fluorophore, wherein the first image data comprises red channel data and green channel data; and
generate, by the one or more processors, a corrected fluorescence image, based on the first image data, wherein generating the fluorescence image comprises subtracting a second component based on one of the green channel data or the red channel data from a first component based on the other of the green channel data or the red channel data.

Example 17. A method for white light and fluorescence imaging using visible light excitation, the method comprising:
illuminating a tissue with a white light illumination light and with an excitation light, wherein the excitation light comprises light within a wavelength range of 380-490 nm;
generating a first visible light image of the tissue based on first image data captured at an image sensor, wherein:
   a blue channel of the first visible light image includes a first white light contribution from reflected white light illumination light and a first excitation contribution from reflected excitation light, and
   the first white light contribution and the first excitation contribution define a first ratio; and
after generating the first visible light image, generating a second visible light image of the tissue based on second image data captured at the image sensor, wherein:
   a white light exposure of the second visible light image is less than or equal to 90% of a white light exposure of the first image or is greater than or equal to 110% of the white light exposure of the first image,
   a blue channel of the second visible light image includes a second white light contribution from the reflected white light illumination light and a second excitation contribution from reflected excitation light, and
   the second white light contribution and the second excitation contribution define a second ratio that is greater than or equal to 95% of the first ratio and less than or equal to 105% of the first ratio.

Example 18. The method of example 17, comprising, before generating the second visible light image:
adjusting a gain of the image sensor to achieve the adjusted white light exposure of the second visible light image.

Example 19. The method of any one of examples 17-18, comprising, before generating the second visible light image:
adjusting an intensity of the white light illumination to achieve the adjusted white light exposure of the second visible light image.

Example 20. The method of any one of examples 17-19, comprising, before generating the second visible light image:
adjusting a white light pulse width of the white light illumination light to achieve the adjusted white light exposure of the second visible light image; and
adjusting an intensity of the excitation light.

Example 21. The method of example 20, comprising, before adjusting the intensity of the excitation light:
calculating the first ratio for the first visible light image; and
determining an amount by which to adjust the intensity of the excitation light based at least in part on the calculated ratio and on an amount by which the white light pulse width is adjusted.

Example 22. The method of any one of examples 20-21, comprising, before adjusting the white light pulse width of the white light illumination, determining an amount by which to adjust the white light pulse width based at least in part on a distance between the image sensor and the tissue.

Example 23. The method of any one of examples 17-22, comprising:
before generating the second visible light image, adjusting a white light pulse width of the white light illumination light to achieve the adjusted white light exposure of the second visible light image; and
wherein generating the second visible light image comprises:
   calculating the first ratio for the first visible light image; and
   applying one or more image processing techniques to the second image data, wherein the one or more image processing techniques are configured based at least in part on the calculated first ratio such that the second white light contribution and the second excitation contribution define the second ratio.

Example 24. The method of any one of examples 17-23, wherein illuminating the tissue with the white light illumination light comprises illuminating with pulsed white light illumination.

Example 25. The method of example 24, wherein illuminating the tissue with the excitation light comprises illuminating with constant excitation light during periods when the white light illumination is on and during periods when the white light illumination is off.

Example 26. The method of any one of examples 17-25, wherein the excitation light is within a wavelength range of 405-415 nm.

Example 27. A system for white light and fluorescence imaging using visible light excitation, the system comprising:
a white light illumination light that illuminates a tissue and an excitation light that illuminates the tissue, wherein the excitation light comprises light within a wavelength range of 380-490 nm; and
an image sensor communicatively coupled to one or more processors, wherein the one or more processors execute instructions stored in memory to cause the system to:
   generate a first visible light image of the tissue based on first image data captured by the image sensor, wherein:
      a blue channel of the first visible light image includes a first white light contribution from reflected white light illumination light and a first excitation contribution from reflected excitation light, and
      the first white light contribution and the first excitation contribution define a first ratio; and
   after generating the first visible light image, generating a second visible light image of the tissue based on second image data captured at the image sensor, wherein:
      a white light exposure of the second visible light image is less than or equal to 90% of a white light exposure of the first image or is greater than or equal to 110% of the white light exposure of the first image,
      a blue channel of the second visible light image includes a second white light contribution from the reflected white light illumination light and a second excitation contribution from reflected excitation light, and
      the second white light contribution and the second excitation contribution define a second ratio that is greater than or equal to 95% of the first ratio and less than or equal to 105% of the first ratio.

Example 28. A non-transitory computer-readable medium storing instructions for white light and fluorescence imaging using visible light excitation, wherein the instructions, when executed by one or more processors of a system comprising a white light illumination light, an excitation light, and an image sensor, cause the system to:
illuminate a tissue with the white light illumination light and with the excitation light, wherein the excitation light comprises light within a wavelength range of 380-490 nm;
generate a first visible light image of the tissue based on first image data captured at the image sensor, wherein:
   a blue channel of the first visible light image includes a first white light contribution from reflected white light illumination light and a first excitation contribution from reflected excitation light, and
   the first white light contribution and the first excitation contribution define a first ratio; and
after generating the first visible light image, generate a second visible light image of the tissue based on second image data captured at the image sensor, wherein:
   a white light exposure of the second visible light image is less than or equal to 90% of a white light exposure of the first image or is greater than or equal to 110% of the white light exposure of the first image,
   a blue channel of the second visible light image includes a second white light contribution from the reflected white light illumination light and a second excitation contribution from reflected excitation light, and
   the second white light contribution and the second excitation contribution define a second ratio that is greater than or equal to 95% of the first ratio and less than or equal to 105% of the first ratio.

Example 29. A method for fluorescence imaging, the method comprising:
illuminating a tissue region with an excitation light, wherein the tissue region comprises tissue and a target fluorophore and wherein the excitation light comprises light within a wavelength range of 380-490 nm;
capturing first image data of the tissue region at one or more image sensors, wherein the first image data comprises red channel data and blue channel data; and
generating a corrected fluorescence image, based on the first image data, wherein generating the fluorescence image comprises subtracting a second component based on the blue channel data from a first component based on the red channel data.

Example 30. The method of example 29, comprising, before subtracting the second component from the first component, applying one or more preprocessing operations to one or both of the red channel data and the blue channel data.

Example 31. The method of any one of examples 29-30, wherein capturing the first image data comprises capturing via a red channel of the one or more image sensors using a first effective gain and capturing via a blue channel of the one or more image sensors using a second effective gain that is less than the first effective gain.

Example 32. The method of example 31, wherein the first effective gain is greater than or equal to 25× the second effective gain.

Example 33. The method of any one of examples 31-32, wherein:
the first image data comprises green channel data; and
the second component is further based on the green channel data.

Example 34. The method of example 33, comprising, before subtracting the second sub-component from the first sub-component, applying one or more preprocessing operations to the green channel data.

Example 35. The method of any one of examples 33-34, wherein capturing the first image data comprises capturing via a green channel of the one or more image sensors using a third effective gain that is less than the first effective gain.

Example 36. The method of example 35, wherein the first effective gain is greater than or equal to 10× the first effective gain.

Example 37. The method of any one of examples 35-36, wherein the second effective gain is equal to the third effective gain.

Example 38. The method of any one of examples 35-37, wherein the third effective is greater than the second effective gain.

Example 39. The method of any one of examples 29-38, wherein:
the steps of illuminating the tissue region with the excitation light and of capturing the first image data are performed during a fluorescence-frame time period; and
the method comprises:
   during a white-light-frame time period different from the fluorescence frame time-period:
      illuminating the tissue region with white-light illumination;
      capturing second image data of the tissue region at the one or more image sensors, wherein the second image data comprises red channel data, green channel data, and blue channel data; and
      generating a white-light image based on the second image data; and
generating a color overlay image based on the corrected fluorescence image and based on the white-light image.

Example 40. The method of any one of examples 29-39, wherein the one or more image sensors comprises a Bayer array image sensor.

Example 41. The method of any one of examples 29-39, wherein the one or more image sensors comprises an RGB prism camera.

Example 42. A system for fluorescence imaging, the system comprising:
an excitation light within a wavelength range of 380-490 nm that illuminates a tissue region, wherein the tissue region comprises tissue and a target fluorophore; and
one or more image sensors communicatively coupled to one or more processors, wherein the one or more processors execute instructions stored in memory to cause the system to:
   capture first image data of the tissue region at one or more image sensors, wherein the first image data comprises red channel data and blue channel data; and
   generate a corrected fluorescence image, based on the first image data, wherein generating the fluorescence image comprises subtracting a second component based on the blue channel data from a first component based on the red channel data.

Example 43. A non-transitory computer-readable medium storing instructions for fluorescence imaging, wherein the instructions, when executed by one or more processors of a system comprising an excitation light within a wavelength range of 380-490 nm, and one or more image sensors, cause the system to:
capture first image data of a tissue region at one or more image sensors, wherein the first image data comprises red channel data and blue channel data; and
   generate a corrected fluorescence image, based on the first image data, wherein generating the fluorescence image comprises subtracting a second component based on the blue channel data from a first component based on the red channel data.

1. A method for fluorescence imaging, the method comprising:
illuminating a tissue region with an excitation light, wherein the tissue region comprises tissue and a target fluorophore and wherein the excitation light comprises light within a wavelength range of 380-490 nm;
capturing first image data of the tissue at an image sensor, wherein the first image comprises an autofluorescence contribution from the tissue and a fluorescence emission contribution from the target fluorophore, wherein the first image data comprises red channel data and green channel data; and
generating a corrected fluorescence image, based on the first image data, wherein generating the fluorescence image comprises subtracting a second component based on one of the green channel data or the red channel data from a first component based on the other of the green channel data or the red channel data.

2. The method of claim 1, wherein the second component comprises a first weight.

3. The method of claim 2, comprising selecting the first weight to minimize intensity of the fluorescence image in one or more regions of the fluorescence image in which the autofluorescence contribution is present but the fluorescence emission contribution is not present.

4. The method of claim 1, comprising, before subtracting the second component from the first component, applying one or more preprocessing operations to one or both of the red channel data and the green channel data.

5. The method of claim 1, wherein the second component is based on the red channel data and the first component is based on the green channel data.

6. The method of claim 1, wherein the second component is based on the green channel data and the first component is based on the red channel data.

7. The method of claim 6, wherein:
the first image data comprises blue channel data; and
the second component is computed by subtracting a second sub-component based on the blue channel data from a first sub-component based on one of the green channel data or the red channel data.

8. The method of claim 7, wherein the second sub-component comprises a second weight.

9. The method of claim 8, comprising selecting the second weight to minimize an effect on the fluorescence image of increasing or decreasing an amount of blue light.

10. The method of claim 7, comprising, before subtracting the second sub-component from the first sub-component, applying one or more preprocessing operations to the blue channel data.

11. The method of claim 1, wherein capturing the first image data comprises exposing a red color channel, a green color channel, and a blue color channel simultaneously.

12. The method of claim 1, comprising displaying an output image, wherein displaying the output is based at least in part on the generated corrected fluorescence image.

13. The method of claim 12, comprising generating the output image, wherein generating the output image comprises:
generating an uncorrected fluorescence image based on the first image data; and
colorizing the corrected fluorescence image based on the uncorrected fluorescence image, wherein the colorization distinguishes the autofluorescence contribution from the fluorescence emission contribution.

14. The method of claim 13, wherein generating the uncorrected fluorescence image comprises summing a fourth component based on one of the green channel data or the red channel data with a third component based on the other of the green channel data or the red channel data.

15. A system for fluorescence imaging, the system comprising:
an excitation light that illuminates a tissue region with an excitation light, wherein the tissue region comprises tissue and a target fluorophore and wherein the excitation light comprises light within a wavelength range of 380-490 nm;
an image sensor that captures first image data of the tissue, wherein the first image comprises an autofluorescence contribution from the tissue and a fluorescence emission contribution from the target fluorophore, wherein the first image data comprises red channel data and green channel data; and
one or more processors, coupled to the image sensor, that execute instructions stored in memory to generate a corrected fluorescence image, based on the first image data, wherein generating the fluorescence image comprises subtracting a second component based on one of the green channel data or the red channel data from a first component based on the other of the green channel data or the red channel data.

16. A non-transitory computer-readable medium storing instructions for fluorescence imaging, wherein the instructions, when executed by one or more processors of a system comprising an excitation light and an image sensor, cause the system to:
illuminate, by the excitation light, a tissue region with an excitation light, wherein the tissue region comprises tissue and a target fluorophore and wherein the excitation light comprises light within a wavelength range of 380-490 nm;
capture, by the image sensor, first image data of the tissue, wherein the first image comprises an autofluorescence contribution from the tissue and a fluorescence emission contribution from the target fluorophore, wherein the first image data comprises red channel data and green channel data; and
generate, by the one or more processors, a corrected fluorescence image, based on the first image data, wherein generating the fluorescence image comprises subtracting a second component based on one of the green channel data or the red channel data from a first component based on the other of the green channel data or the red channel data.
